(19) United States
(12) Patent Application Publication
(10) Pub. No.: US 2004/0137419 A1
(43) Pub. Date: Jul. 15, 2004

(54) Methods for removing microbicidal compounds from compositions

(75) Inventors: Andrei Purmal, Waltham, MA (US); David Tabatadze, Worcester, MA (US)

Correspondence Address: John R. Van Amsterdam, Ph.D., Wolf, Greenfield & Sacks, P.C., 600 Atlantic Avenue, Boston, MA, 02210, US

(73) Assignee: V.I. Technologies, Inc., Watertown, MA

(21) Appl. No.: 10/679,933

(22) Filed: Oct. 6, 2003

Related U.S. Application Data
(60) Provisional application No. 60/440,287, filed on Jan. 15, 2003.

(51) Int. Cl. A01N 1/02

(52) U.S. Cl. 435/2

(57) ABSTRACT

Methods for removal of positively charged microbicidal compounds from biological compositions are provided.

### RELATED APPLICATIONS

This application claims priority under 35 U.S.C. § 119(e) from U.S. provisional patent application serial No. 60/440,287, filed Jan. 15, 2003, the entire disclosure of which is incorporated herein by reference.

### FIELD OF THE INVENTION

The invention relates to methods for removing positively charged microbicidal compounds from treated compositions, and products resulting from the methods.

### BACKGROUND OF THE INVENTION

The transmission of viral diseases (e.g., hepatitis A, B, and C, acquired immunodeficiency syndrome, and cytomegalovirus infections) and other pathogenic organisms by biological products including blood or blood products is a significant problem in medicine. Screening donor blood for viral markers can help reduce the transmission of viruses to recipients, but many screening methods are directed to only a few discrete viruses and are therefore incomplete or less than 100% sensitive. Emerging viruses and viruses entering the blood supply (e.g., West Nile virus), for which screening tests have not yet been developed pose another problem. Furthermore, other biological compositions, such as mammalian and hybridoma cell lines, products of cell lines, milk, colostrum, and sperm, can contain infectious viruses as well. It is therefore important to inactivate viruses and other pathogenic organisms contained in donor blood, blood products, or other biological compositions.

A number of positively charged microbicidal compounds that are capable of inactivating pathogens in biological compositions such as blood have been developed. For example, ethyleneimine monomer and ethyleneimine oligomers are very effective viral inactivating agents. These agents are themselves toxic, and must therefore be rendered non-toxic or removed from the biological composition before a composition, such as blood, is used clinically.

To inactivate a microbicidal compound, a quenching agent typically is added to inactivate the microbicidal compound that remains after pathogen inactivation has taken place. The end result is a biological composition that is relatively free of infectious agents, but that is contaminated with quenched microbicidal compound and with quenching agent.

Removal of positively charged microbicidal compounds from a biological composition such as a red blood cell concentrate (RBCC) typically involves washing the treated RBCC extensively using either manual or automated cell washing procedures. While this methodology does remove some of the microbicidal compounds, the procedure for cell washing consumes a great deal of reagents and time, and generates a significant quantity of waste product.

Therefore, there is a need for a more efficient method for removing positively charged microbicidal compounds from treated biological compositions.

### SUMMARY OF THE INVENTION

It has been discovered that the use of cation exchanger compounds, such as resins, can substantially reduce the level of positively charged microbicidal compounds in biological compositions that have been treated with such microbicidal compounds. The invention provides methods for removing microbicidal compounds, as well as products resulting from the methods that have unexpectedly superior storage properties as compared to biological compositions treated with automated washing methods to reduce microbicidal compounds.

According to a first aspect of the invention, methods for removing a positively charged microbicidal compound and/or degradation products or derivatives thereof from a composition are provided. The methods include contacting a composition with a cation exchange resin, under conditions and for a time sufficient to allow a positively charged microbicidal compound and/or degradation products or derivatives thereof in the composition to bind to the cation exchange resin, and separating the positively charged microbicidal compound and/or degradation products or derivatives thereof bound to the cation exchange resin from the composition.

According to a second aspect of the invention, methods for removal of an intracellular positively charged microbicidal compound and/or degradation products or derivatives thereof from a cell treated with a positively charged microbicidal compound. The methods include contacting a composition containing a cell treated with a positively charged microbicidal compound with a cation exchange resin, under conditions and for a time sufficient to allow the positively charged microbicidal compound and/or degradation products or derivatives thereof to bind to the cation exchange resin. In certain embodiments of this aspect of the invention, the methods also include a step of separating the positively charged microbicidal compound and/or degradation products or derivatives thereof bound to the cation exchange resin from the cell.

In certain embodiments of the foregoing aspects of the invention, the positively charged microbicidal compound is positively charged at physiological pH.

In some preferred embodiments of the foregoing aspects of the invention, the cation exchange resin is not contained within a matrix and/or the positively charged microbicidal compound is an aziridino compound. More preferably, the aziridino compound is an ethyleneimine oligomer.

In other preferred embodiments of the foregoing aspects of the invention, the composition is a blood product. More preferably, the method does not substantially change the biological properties of the treated blood product relative to untreated blood product. Still more preferably, the biological properties of the treated blood product are not substantially changed after storage at 4° C. relative to untreated blood product. In some of these embodiments, the blood product is a composition comprising red blood cells. Preferably, the method results in a level of hemolysis of the red blood cells that is no greater than cell washing or the surface of the cation exchange resin particles does not induce substantial hemolysis of the red blood cells.

In further embodiments of the foregoing aspects of the invention, the methods also include washing the blood product after removal of the positively charged microbicidal compound bound to the cation exchange resin. In other embodiments of the foregoing aspects of the invention, the methods also include washing the blood product prior to contacting the composition with a cation exchange resin.

The cation exchange resin has certain preferred properties. The cation exchange resin is a strongly acidic cation exchange resin in some embodiments of the foregoing aspects of the invention; preferably the cation exchange resin comprises sulfonic groups. In a particularly preferred embodiment, the cation exchange resin is DOWEX™ 50W×8. In other embodiments of the foregoing aspects of the invention, the diameter of the cation exchange resin particles is at least about 100 microns, preferably between about 150 microns and about 300 microns. In still other embodiments of the foregoing aspects of the invention, the cation exchange resin particles are substantially non-breakable under moderate mechanical stress in dry conditions or suspensions, and/or are compatible with water miscible solvents (preferably the cation exchange resin particles do not dissolve or degrade when contacted with water miscible solvents), and/or do not create fine particles under moderate mechanical stress.

In further preferred embodiments, the cation exchange resin particles have a cation exchange capacity of at least about 1 meq/ml, preferably at least about 2 meq/ml, more preferably at least about 3 meq/ml, and still more preferably at least about 5 meq/ml. The cation exchange resin particles do not leach toxic components into water based media or blood products, and/or are sterilized by gamma or thermal sterilization in still further embodiments of the invention. In other embodiments, the cation exchange resin particles have substantially no pores.

In other embodiments of the foregoing aspects of the invention, the method is performed under pH conditions of from about pH 4 to about pH 14, preferably from about pH 6 to about pH 8, and more preferably at about pH 7.

In still other embodiments of the foregoing aspects of the invention, the method is performed using a column format, in which the step of contacting a composition with a cation exchange resin is performed by flowing the composition into a column packed with the cation exchange resin, and the step of separating the positively charged microbicidal compound and/or degradation products or derivatives thereof bound to the cation exchange resin from the composition is performed by flowing the composition out of the column. In these embodiments, the column preferably is run by gravity or by moderate pressure with a flow rate of less than about 50 m/min, more preferably the flow rate is less than about 10 ml/min, and still more preferably the flow rate is less than about 1 ml/min.

In still other embodiments of the foregoing aspects of the invention, the amount of positively charged microbicidal compound in the composition is reduced by at least about 2 logs, preferably by at least about 3 logs, more preferably by at least about 4 logs, and still more preferably by at least about 5 logs.

The temperature of the foregoing methods can be varied also, typically within the range of about 20° C. to about 37° C. Thus, in certain embodiments, the methods are performed at a temperature of at least about 20° C., preferably at least about 25° C., more preferably at least about 27° C., and still more preferably at least about 30° C.

The size and shape of a column used in the foregoing methods also can be varied to suit particular applications. In some embodiments of the invention, the dimensions of the column expressed as a ratio of diameter:length are about 1:5 or less. Preferably the dimensions of the column expressed as a ratio of diameter:length are about 1:10 or less, and more preferably the ratio is about 1:20 or less.

In one particular preferred embodiment, the foregoing methods are performed in a column format, the flow rate is from about 1 ml/min to about 2 ml/min, the temperature is about 25° C., and the dimensions of the column expressed as a ratio of diameter:length are about 1:10 or less. Various other combinations of the embodiments described herein are also possible and are embraced by the invention.

According to certain embodiments of the invention, the methods are performed in a batch format. In preferred embodiments, the step of separating the positively charged microbicidal compound and/or degradation products or derivatives thereof bound to the cation exchange resin from the composition is performed by filtration of the composition to remove the cation exhange resin, or is performed by centrifugation of the composition to remove the cation exchange resin. Combinations of filtration and centrifugation also can be performed.

In other embodiments of the batch methods, the step of contacting the composition with the cation exchange resin is performed by adding the cation exchange resin in a permeable container to the composition. In these embodiments, the step of separating the positively charged microbicidal compound and/or degradation products or derivatives thereof bound to the cation exchange resin from the composition preferably is performed by removing the permeable container from the composition.

In further embodiments of the batch methods, the step of contacting the composition with the cation exchange resin is performed by adding the composition to a container that contains cation exchange resin. In these embodiments, the step of separating the positively charged microbicidal compound and/or degradation products or derivatives thereof bound to the cation exchange resin from the composition preferably is performed by removing the composition from the container or by removing the resin and bound positively charged microbicidal compound and/or degradation products or derivatives thereof from the container.

In still other embodiments of the batch methods, the step of contacting the composition with the cation exchange resin is performed by adding the cation exchange resin to the composition. In these embodiments, the step of separating the positively charged microbicidal compound and/or degradation products or derivatives thereof bound to the cation exchange resin from the composition is performed by filtration of the composition to remove the cation exhange resin, and/or by centrifugation of the composition to remove the cation exchange resin.

In preferred embodiments of the foregoing batch methods, steps of contacting the composition with the cation exchange resin and separating the positively charged microbicidal compound and/or degradation products or derivatives thereof bound to the cation exchange resin from the composition are performed at least twice.

In preferred embodiments of the foregoing batch methods, the composition comprises cells, and the methods also include washing the cells (prior to or after performing the batch methods); preferably the cells are washed using an automated cell washer.

In certain embodiments of the foregoing batch methods, the concentration of microbicidal compound is reduced by at least about 50%, preferably by at least about 1 log, more preferably by at least about 2 logs, still more preferably by at least about 3 logs, and yet more preferably by at least about 4 logs.

According to a third aspect of the invention, blood products treated according to any of the foregoing methods are provided.

According to a fourth aspect of the invention, containers for blood products are provided. The containers include cation exchange resin. In certain embodiments, the cation exchange resin is loose in the container, while in other embodiments, the cation exchange resin is contained within a permeable enclosure. Preferably the cation exchange resin is a strong cation exchanger.

In certain embodiments, the amount of cation exchange resin is sufficient to reduce the amount of a positively charged microbicidal compound and/or degradation products or derivatives thereof in the blood product after contact with the resin. Preferably, the amount of cation exchange resin in the container is sufficient to reduce the amount of the positively charged microbicidal compound and/or degradation products or derivatives thereof by at least about 50%, more preferably by at least about 1 log, more preferably by at least about 2 logs, still more preferably by at least about 3 logs, and yet more preferably by at least about 4 logs.

In some embodiments, the positively charged microbicidal compound is an aziridino compound; preferably the aziridino compound is an ethyleneimine oligomer.

Other embodiments of the invention will be clear from the following description of the invention.

### BRIEF DESCRIPTION OF THE DRAWINGS

FIG. 1 is a graph illustrating the effect of the amount of DOWEX 50W×8 resin on PEN110 removal from RBCC as shown by the post column level of PEN110.

FIG. 2 is a graph illustrating the effect of the amount of DOWEX 50W×8 resin on PEN110 removal from RBCC as shown by the level of PEN110 following manual washing or automated washing.

FIG. 3 is a graph showing the effect of RBCC flow rate of on PEN110 removal as shown by the post column level of PEN110.

FIG. 4 is a graph showing the effect of RBCC flow rate of on PEN110 removal as shown by the level of PEN110 following manual washing or automated washing.

FIG. 5 is a graph of the effect of temperature on PEN110 removal from RBCC as shown by the post column level of PEN110.

FIG. 6 is a graph of the effect of temperature on PEN110 removal from RBCC as shown by the level of PEN110 following manual washing or automated washing.

FIG. 7 is a graph illustrating the effect of column configuration on PEN110 removal from RBCC as shown by the post column level of PEN110.

FIG. 8 is a graph of the hemolysis level during the storage of RBCC purified using cation exchange (test) or automated washing (control).

FIG. 9 is a graph of ATP level in RBC during the storage of RBCC purified using cation exchange (test) or automated washing (control).

FIG. 10 is a graph of percent methemoglobin formation during the storage of RBCC purified using cation exchange (test) or automated washing (control).

FIG. 11 is a graph of hematocrit of RBCC during the storage of RBCC purified using cation exchange (test) or automated washing (control).

FIG. 12 is a graph of extracellular K⁺ in RBCC during the storage of RBCC purified using cation exchange (test) or automated washing (control).

FIG. 13 is a graph of intracellular K⁺ in RBCC during the storage of RBCC purified using cation exchange (test) or automated washing (control).

FIG. 14 is a graph of mean corpuscular volume (MCV) of RBC during the storage of RBCC purified using cation exchange (test) or automated washing (control).

FIG. 15 is a graph of pH of RBCC during the storage of RBCC purified using cation exchange (test) or automated washing (control).

FIG. 16 is a graph of total hemoglobin of RBCC during the storage of RBCC purified using cation exchange (test) or automated washing (control).

FIG. 17 is a graph of mean cellular hemoglobin concentration (MCHC) in RBC during the storage of RBCC purified using cation exchange (test) or automated washing (control).

FIG. 18 is a graph of osmolality of RBCC during the storage of RBCC purified using cation exchange (test) or automated washing (control).

FIG. 19 is a graph of RBC count in RBCC during the storage of RBCC purified using cation exchange (test) or automated washing (control).

FIG. 20 is a schematic diagram of the experimental procedure used in the Example.

FIG. 21 is a graph illustrating the effect of the strong and weak cation exchange resins on PEN110 removal from RBCC as shown by the post column level of PEN110.

FIG. 22 is a graph illustrating the effect of removal of PEN110 from RBCC using fresh portions of Dowex 50W×8 resin.

### DETAILED DESCRIPTION OF THE INVENTION

The invention provides methods for removal of positively charged microbicidal compounds from compositions. As used herein, a “microbicidal compound” is a compound, preferably a small organic molecule, which inactivates or kills a microbe. Microbicidal compounds include antiviral agents, antibacterial agents, antifungal agents, antiprotozoal agents, and the like. A “microbicidal compound” and other similar terms are used as equivalent terms herein.

The present methods involve contacting a biological composition that has been treated with a positively charged microbicidal compound with a cation exchange resin. The methods are performed under conditions and for a time sufficient to allow a positively charged microbicidal compound and/or degradation products or derivatives thereof in the composition to bind to the cation exchange resin. The methods also involve separating the positively charged microbicidal compound and/or degradation products or derivatives thereof bound to the cation exchange resin from the composition.

The methods can be performed in batch mode or in flow mode, such as in a column format. When a column is used, flow of treated biological compositions and reagents (e.g., washing reagents) is typically gravity flow, but can be flow created by the application of pressure to the column. As used herein, a “column” refers broadly to a chamber or device that includes cation exchange resin that will remove positively charged microbicidal compound and/or degradation products or derivatives thereof. Accordingly, column includes cartridges, containers, and other means for housing such material.

In batch mode, a biological composition treated with a microbicidal compound is contacted with a cation exchange resin under conditions and for a time sufficient to remove microbicidal compound, and then the resin is separated from the composition after removing the microbicidal compound. Separation of the resin can be achieved using a variety of well known physical separation means, such as filtration, centrifugation, magnetic separation, and the like. Preferably the batch removal of microbicidal compound is repeated to further reduce the concentration of microbicidal compound in the biological composition.

The resin may also be contained in an enclosure for contacting the biological composition. Preferably such an enclosure is permeable, such that diffusion of the microbicidal compound into the enclosure for contact with the resin is not hindered. In a preferred embodiment, the resin is enclosed in a membrane that is permeable to small molecules such as microbicidal compounds (analogous to a tea bag retaining the tea leaves but permitting flow of water in and out). After contact with the biological composition under conditions and for a time sufficient to remove microbicidal compound, the enclosed resin can simply be removed from the biological composition (much as a tea bag is removed from tea after steeping). Additional measures of cation exchange resin can then be added to remove remaining microbicidal compound and further reduce the concentration of microbicidal compound in the biological composition.

Acceptable properties of cation exchange resins are selected from the following: resin bead size of 100 micron or larger; non-breakable under moderate mechanical stress; compatible with all water miscible solvents; the surface of resin particles or beads does not induce significant amounts of hemolysis of red blood cells; high cation exchange capacity (about 1-4 meq/ml); no toxic leachables into water based media or blood products; suitable for gamma or thermal sterilization; does not dissolve or degrade in water-based media; does not change in vitro quality of blood products during contact; does not change in vitro quality of blood products after removal of particles or beads and storage at 4° C.

Preferred properties of cation exchange resins are selected from the following: resin bead size of about 300-1200 microns; non-breakable under mechanical stress in dry conditions or suspensions; do not create fine particles; compatible with all solvents; easily dried and washed; suitable for low/high temperatures (0-150° C.); suitable for exposure to conditions of pH 1-14; stable at room temperature at least for one year; smooth surface; resin particles or beads do not induce hemolysis of red blood cells; high cation exchange capacity (at least about 2-4 meq/ml); no toxic leachables into water based media; can be sterilized by gamma irradiation or heat; does not dissolve or degrade in water based media; does not change in vitro quality of blood products; does not change in vitro quality of blood products during the storage at 4° C. before or after contact with cation exchangers to blood product; and allows RBCC to pass through a column or cartridge with gravity flow.

The properties of the cation exchange particles or beads are selected based on the purpose for which they are used. For example, for greater removal of positively charged microbicidal compounds, the bead size will be smaller, and for less removal of compound, the bead size will be larger. Typically the bead size will vary by ±100 microns around a selected size, e.g., 400±100 microns. Similarly, when used in a column or cartridge format, the column or cartridge size and flow rate can be varied according to the requirements of the removal process. For example, for fast removal of positively charged microbicidal compounds, the skilled artisan can use a column with a wider diameter and a relatively faster flow rate. For more complete removal of positively charged microbicidal compounds, a narrower and/or longer column can be used, and/or a slower flow rate through the column.

Certain properties of cation exchange resins are unacceptable, including the following: resin bead size of less than 50 microns; very rigid (i.e., breakable under moderate mechanical stress); non-compatible with water based media; easily breakable in dry conditions; not suitable for exposure to conditions of pH 4-14; not stable at room temperature; resin particles or beads contain sharp edges; produce toxic leachables during the incubation in water-based buffers and blood products; chemically reacts with blood products; dissolves or degrades in blood products; changes in vitro quality of blood products; changes in vitro quality of blood products during storage at 4° C.; unstable during thermal and/or gamma sterilization; does not allow RBCC to pass through a column or cartridge; very low cation exchange capacity (i.e., lower than 0.2 meq/ml).

The resin used in the invention is negatively charged due to the presence of one or more types of cation exchange functional groups on the resin. Cation exchange functional groups are atoms, molecules or chemical groups that at an appropriate pH are negatively charged. Cation exchange functional groups include but are not limited to sulfonic, sulfate, carboxyl, thiophosphate, dithiophosphate, trithiophosphate, thiosulfate, phosphate, phosphonic and aminophosphonic groups. The cation exchange resins useful in accordance with the invention are preferably strong cation exchangers, although weak cation exchange resin also will work. Strong acid cation exchange resins that can be used in accordance with the invention preferably are those composed of sulfonic acid functional groups attached to a styrene divinylbenzene copolymer lattice. Examples of these resins include DOWEX™ 50W×8 (Dow Chemical, Midland Mich.) and AG 50W-×8 (Bio-Rad, Hercules, Calif.). For these resins, the term “50W” indicates a strong cation exchanger, and the term “×8” or the like indicates the degree of resin crosslinkage, i.e., the percentage of divinylbenzene in the resin copolymer. Thus, DOWEX 50W×8 is a strong acid cation resin containing 8% divinylbenzene. Other strong cation exchange resins known to the person of skill in the art, such as AMBERLITE™ IR120 Na (Rohm and Haas, Philadelphia, Pa.), also can be used. A listing of commercially available cation exchange resins useful in accordance with the invention is presented in the following table.

Table of commercially available cation exchangers:

| | | Matrix | Type of | |
|---|---|---|---|---|
| Resin | Manufacturer | composition | exchanger | Functional group |
| SST-60 | Purolite | Polystyrene- | Strong cation | Sulfonic |
| | | DVB | exchanger | |
| C100 | Purolite | Polystyrene- | Strong cation | Sulfonic |
| | | DVB | exchanger | |
| C110H | Purolite | Polystyrene- | Strong cation | Sulfonic |
| | | DVB | exchanger | |
| C100E | Purolite | Polystyrene- | Strong cation | Sulfonic |
| | | DVB | exchanger | |
| C100x10 | Purolite | Polystyrene- | Strong cation | Sulfonic |
| | | DVB | exchanger | |
| C145 | Purolite | Polystyrene- | Strong cation | Sulfonic |
| | | DVB | exchanger | |
| C147 | Purolite | Polystyrene- | Strong cation | Sulfonic |
| | | DVB | exchanger | |
| C150 | Purolite | Polystyrene- | Strong cation | Sulfonic |
| | | DVB | exchanger | |
| C150H | Purolite | Polystyrene- | Strong cation | Sulfonic |
| | | DVB | exchanger | |
| C155 | Purolite | Polystyrene- | Strong cation | Sulfonic |
| | | DVB | exchanger | |
| C160 | Purolite | Polystyrene- | Strong cation | Sulfonic |
| | | DVB | exchanger | |
| C160H | Purolite | Polystyrene- | Strong cation | Sulfonic |
| | | DVB | exchanger | |
| C104 | Purolite | Polyacrylic | Weak cation | Carboxyl |
| | | | exchanger | |
| C106 | Purolite | Polyacrylic | Weak cation | Carboxyl |
| | | | exchanger | |
| C107E | Purolite | Polymethacrylic | Weak cation | Carboxyl |
| | | | exchanger | |
| C115 | Purolite | Polyacrylic | Weak cation | Carboxyl |
| | | | exchanger | |
| S-940 | Purolite | Polystyrene- | Weak cation | Aminophosphonic |
| | | DVB | exchanger | |
| S-950 | Purolite | Polystyrene- | Weak cation | Aminophosphonic |
| | | DVB | exchanger | |
| S100 | Bayer-Lewatit | Polystyrene- | Strong cation | Sulfonic |
| | | DVB | exchanger | |
| S100LF | Bayer-Lewatit | Polystyrene- | Strong cation | Sulfonic |
| | | DVB | exchanger | |
| S110 | Bayer-Lewatit | Polystyrene- | Strong cation | Sulfonic |
| | | DVB | exchanger | |
| SP112 | Bayer-Lewatit | Polystyrene- | Strong cation | Sulfonic |
| | | DVB | exchanger | |
| SP120 | Bayer-Lewatit | Polystyrene- | Strong cation | Sulfonic |
| | | DVB | exchanger | |
| CNP-80 | Bayer-Lewatit | Polyacrylic | Weak cation | Carboxyl |
| | | | exchanger | |
| CNP/LF | Bayer-Lewatit | Polyacrylic | Weak cation | Carboxyl |
| | | | exchanger | |
| HCR-S(E) | Dow | Polystyrene- | Strong cation | Sulfonic |
| | | DVB | exchanger | |
| HCR-S(E)S | Dow | Polystyrene- | Strong cation | Sulfonic |
| | | DVB | exchanger | |
| HGR/HGR- | Dow | Polystyrene- | Strong cation | Sulfonic |
| W2/C 10 | | DVB | exchanger | |
| MSC-1/88/CM-12 | Dow | Polystyrene- | Strong cation | Sulfonic |
| | | DVB | exchanger | |
| 50W | Dow | Polystyrene- | Strong cation | Sulfonic |
| | | DVB | exchanger | |
| CM/15/16 | Dow | Polystyrene- | Strong cation | Sulfonic |
| | | DVB | exchanger | |
| MWC-1 | Dow | Polyacrylic | Weak cation | Carboxyl |
| | | | exchanger | |
| CCR-2/3 | Dow | Polyacrylic | Weak cation | Carboxyl |
| | | | exchanger | |
| MWC-2 | Dow | Polyacrylic | Weak cation | Carboxyl |
| | | | exchanger | |
| MAC-3 | Dow | Polymethacrylic | Weak cation | Carboxyl |
| | | | exchanger | |
| SK 1B | Mitsubishi- | Polystyrene- | Strong cation | Sulfonic |
| | Diaion | DVB | exchanger | |
| SK110 | Mitsubishi- | Polystyrene- | Strong cation | Sulfonic |
| | Diaion | DVB | exchanger | |
| PK 216 | Mitsubishi- | Polystyrene- | Strong cation | Sulfonic |
| | Diaion | DVB | exchanger | |
| PK228 | Mitsubishi- | Polystyrene- | Strong cation | Sulfonic |
| | Diaion | DVB | exchanger | |
| WK 40 | Mitsubishi- | Polyacrylic | Weak cation | Carboxyl |
| | Diaion | | exchanger | |
| WK 20 | Mitsubishi- | Polyacrylic | Weak cation | Carboxyl |
| | Diaion | | exchanger | |
| WK 10/11 | Mitsubishi- | Polyacrylic | Weak cation | Carboxyl |
| | Diaion | | exchanger | |
| Diphonix | ELCroM | Polystyrene- | Strong cation | Sulfonic and |
| | | DVB | exchanger | diphosphonic |
| CG 8 | Resintech | Polystyrene- | Strong cation | Sulfonic |
| | | DVB | exchanger | |
| CG 8H | Resintech | Polystyrene- | Strong cation | Sulfonic |
| | | DVB | exchanger | |
| SAC MP | Resintech | Polystyrene- | Strong cation | Sulfonic |
| | | DVB | exchanger | |
| WAC MP | Resintech | Polyacrylic | Weak cation | Carboxyl |
| | | | exchanger | |
| IR 120 | Rohm & Haas - | Polystyrene- | Strong cation | Sulfonic |
| | Amberlite | DVB | exchanger | |
| SR 1L | Rohm & Haas - | Polystyrene- | Strong cation | Sulfonic |
| | Amberlite | DVB | exchanger | |
| IR 122 | Rohm & Haas - | Polystyrene- | Strong cation | Sulfonic |
| | Amberlite | DVB | exchanger | |
| Amb 252 | Rohm & Haas - | Polystyrene- | Strong cation | Sulfonic |
| | Amberlite | DVB | exchanger | |
| Amb 200 | Rohm & Haas - | Polystyrene- | Strong cation | Sulfonic |
| | Amberlite | DVB | exchanger | |
| IRC 76/84 | Rohm & Haas - | Polyacrylic | Weak cation | Carboxyl |
| | Amberlite | | exchanger | |
| IRC 86 | Rohm & Haas - | Polyacrylic | Weak cation | Carboxyl |
| | Amberlite | | exchanger | |
| IRC 50 | Rohm & Haas - | Polyacrylic | Weak cation | Carboxyl |
| | Amberlite | | exchanger | |
| C 249/C298 | Sybron-Ionac | Polystyrene- | Strong cation | Sulfonic |
| | | DVB | exchanger | |
| C250/C299 | Sybron-Ionac | Polystyrene- | Strong cation | Sulfonic |
| | | DVB | exchanger | |
| CFP110 | Sybron-Ionac | Polystyrene- | Strong cation | Sulfonic |
| | | DVB | exchanger | |
| C360 | Sybron-Ionac | Polystyrene- | Strong cation | Sulfonic |
| | | DVB | exchanger | |
| CC | Sybron-Ionac | Polyacrylic | Weak cation | Carboxyl |
| | | | exchanger | |
| CNN | Sybron-Ionac | Polyacrylic | Weak cation | Carboxyl |
| | | | exchanger | |
| PL-SO3H | Polymer | Polystyrene- | Strong cation | Sulfonic |
| | Laboratories | DVB | exchanger | |
| MP-TsOH | Argonaut | Polystyrene- | Strong cation | Sulfonic |
| | | DVB | exchanger | |
| CM-650 S | TosoHaas | Methacrylic | Weak cation | Carboxyl |
| | | | exchanger | |
| CM-650 M | TosoHaas | Methacrylic | Weak cation | Carboxyl |
| | | | exchanger | |
| CM-650 C | TosoHaas | Methacrylic | Weak cation | Carboxyl |
| | | | exchanger | |
| SP-650S | TosoHaas | Methacrylic | Strong cation | Sulfonic |
| | | | exchanger | |
| SP-650M | TosoHaas | Methacrylic | Strong cation | Sulfonic |
| | | | exchanger | |
| SP-650C | TosoHaas | Methacrylic | Strong cation | Sulfonic |
| | | | exchanger | |
| SP-550C | TosoHaas | Methacrylic | Strong cation | Sulfonic |
| | | | exchanger | |
| SC5080 | Advanced | Polystyrene- | Weak cation | Carboxyl |
| | ChemTech | DVB | exchanger | |
| Macro-prep | Bio-Rad | Methacrylic | Strong cation | Sulfonic |
| high S | | | exchanger | |
| Macro-prep | Bio-Rad | Methacrylic | Weak cation | Carboxyl |
| high CM | | | exchanger | |

The methods of the invention also can be practiced using alternative physical forms that provide cation exchange properties. For example, a variety of materials other than the styrene divinylbenzene copolymer particles or beads described above can be used as a solid support for cation exchanger functional groups (preferably sulfonic acid functional groups). Functional groups can be attached directly to a solid support, or they can be attached to the solid support through linkers. Examples of solid support materials include natural and synthetic polymers (e.g., polyvinyl chloride, polytetrafluoroethylene), polymers produced by combinatorial chemistry, nylons (e.g., DACRON™), solids with negative charge on the surface, polyacrylamide pads deposited on solid surfaces, silicon, silicon-glass, glass and combinations thereof. The solid supports may be in any form or shape suitable for use in the invention. For example, the solid supports may be shaped as beads, rods, or films; alternatively, the solid supports may be in the form of permeable and semi-permeable membranes. The solid support may be contained within a filtration device, such as a column or a cartridge. The solid support also may be contained within a device that is permeable and configured to remain in contact with a biological composition (e.g., in a blood product storage container or transfer container) or to be readily removed from a biological composition after contact with the a biological composition to remove a microbicidal compound.

As an example of the methods of the invention, a cation exchange resin can be used as follows. A viral inactivating agent, such as an ethyleneimine oligomer, is added to a biological composition, such as a blood product, as described in U.S. Pat. No. 6,136,586. At the end of the time necessary for viral inactivation, the biological composition is contacted with a solid support containing cation exchange functional groups, preferably a strong cation exchange resin. The step of contacting can be carried out by passing the composition through a filtration device, such as a column that contains cation exchange resin. Alternatively, the support can be added to the treated biological composition for batch removal of microbicidal compounds, such as in the form of particles; these particles can be removed, for example, by filtration. In a preferred embodiment of batch processing, the support (e.g., resin particles) is preferably contained within a removable container that is permeable to microbicidal compounds, such as a pouch that is analogous to a teabag.

The cation exchanger functional groups (e.g., sulfonic acid functional groups) interact with the positively charged ethyleneimine compounds, more specifically the primary amino group(s) of the ethyleneimine compounds. When the contact between the cation exchange resin and the biological composition is terminated, e.g., by elution from a column, the ethyleneimine compounds are removed as well from the biological composition. The resulting composition is a biological composition that is substantially free of infectious viruses and positively charged ethyleneimine microbicidal compounds.

Preferred conditions for the use of a column format in the methods of the invention include the following: a flow rate of less than about 50 ml/min, preferably in the range of about 1 ml/min to about 10 ml/min, i.e., about 1 ml/min, about 2 ml/min, about 3 ml/min, about 4 ml/min, about 5 ml/min, about 6 ml/min, about 7 ml/min, about 8 ml/min, about 9 ml/min, about 10 ml/min; a temperature of at least about 20° C., preferably in a range between about 20° C. and about 37° C., i.e., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or about 37° C.

Preferred column configurations include columns having dimensions expressed as a ratio of diameter:length that are about 1:5 or less, more preferably 1:10 or less, and still more preferably 1:20 or less, e.g., 1:25, 1:50, 1:100, 1:150, 1:200, 1:250, 1:300, etc. preferred lengths of columns are at least 10 cm, more preferably at least 20 cm, still more preferably at least 25 cm, still more preferably at least 30 cm, still more preferably at least 50 cm, still more preferably at least 75 cm, still more preferably at least 100 cm, still more preferably at least 150 cm, still more preferably at least 200 cm, and so on.

As a result of the methods of the invention, the amounts of positively charged microbicidal compounds in treated biological compositions can be substantially reduced. Thus, the amount of positively charged microbicidal compound in the composition can be reduced by at least about 2 logs, preferably by at least about 2.5 logs, more preferably by at least about 3 logs, more preferably by at least about 4 logs, and most preferably by at least about 5 logs. Preferably the methods result in a reduction of positively charged microbicidal compounds in the composition to less than 50 ng/mL.

As used herein, a “biological composition” means a composition containing cells or biopolymers. Cell-containing compositions including, for example, whole blood, red blood cell concentrates, platelet concentrates, leukocyte concentrates, blood cell proteins, blood plasma protein fractions, purified blood proteins, serum, semen, mammalian colostrum and milk, placental extracts, products of fermentation, ascites fluid, and products produced in cell culture by normal or transformed cells (e.g., via recombinant DNA or monoclonal antibody technology). A “biopolymer” or “biological molecule” means any class of organic molecule normally found in living organisms including, for example, nucleic acids, polypeptides, post-translationally modified proteins (e.g., glycoproteins), polysaccharides and lipids. Biopolymer-containing compositions include, for example, blood cell proteins, blood plasma, blood plasma fractionation precipitates, blood plasma fractionation supernatants, cryoprecipitates, cryosupernatants, or portion or derivative thereof, or serum, or a non-blood product produced from normal or transformed cells (e.g., via recombinant DNA technology). Biological compositions can be cell-free.

The methods of the invention are useful for reduction of the amount of positively charged microbicidal compounds in treated biological compositions. The methods are applicable to any positively charged microbicidal compound. Examples of positively charged microbicidal compounds are compounds utilizing aziridino chemistry. The aziridino compounds include in certain embodiments aziridino compounds with an alkyl chain, such as ethyleneimine oligomers, which are positively charged electrophilic molecules chemically related to binary ethyleneimine that has selective reactivity with nucleic acids. As used herein, an “ethyleneimine oligomer” can refer to an ethyleneimine dimer, an ethyleneimine trimer, an ethyleneimine tetramer or derivative thereof. Methods for synthesis of aziridino compounds, particularly ethyleneimine oligomers, are provided, for example, in U.S. Pat. No. 6,215,003 and Kostyanovskii et al., “Oligomer of Aziridines and N-β-Aziridinoethylamides,” Institute of Chemical Physics of the Academy of Sciences of the U.S.S.R. Moscow. Izvestiya Akademii Nauk SSSR, Seriya Khimicheskaya 11:2566-2575 (1988).

The aziridino compounds have a method of action that includes disruption of nucleic acid replication and/or transcription to achieve desirable biological effects. The electrostatic binding of positively charged aziridino compounds such as ethyleneimine oligomers to nucleic acid molecules results in a covalent interaction of the aziridino group with nucleophilic groups of DNA or RNA, predominantly the N-7 position of guanine. Covalent modification of nucleic acid bases can cause loss of the base, i.e., formation of abasic sites, or even strand breaks. Abasic sites and strand breaks produced by ethyleneimine oligomer adducts with nucleic acids act as potent stop signals for nucleic acid polymerases. Accordingly, the modified nucleic acids can not serve as templates for replication or transcription.

Aziridino-containing compounds useful in the methods of the invention preferably contain a moiety having the formula (I):

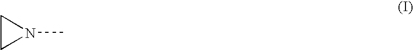

In this three-membered ring, the two carbons are preferably unsubstituted (i.e., they contain hydrogens), but they can be substituted with aliphatic or aromatic hydrocarbon moieties, each containing between one and four carbon atoms, inclusive.

Various aziridino compounds are disclosed in U.S. Pat. Nos. 6,093,564 and 6,136,586, and in U.S. application No. 60/378,184, filed on May 6, 2002, entitled Methods and Compositions for the Modification of Nucleic Acids, the entire disclosures of which are incorporated by reference. Of course, if desired, other microbicidal compounds that are not aziridino-containing compounds can be removed from biological compositions after use as described in the present invention.

The use of the aforementioned aziridino compounds or other microbicidal compounds typically involves contacting a biological composition with an amount of an aziridino compound (and/or other microbicidal compounds) effective to inactivate or kill microbes in the biological composition. It is desirable to remove the microbicidal compounds (and any degradation products or derivatives thereof) from the treated biological composition, to reduce the amount of the microbicidal compounds which typically are toxic molecules.

The following example is provided for the purpose of illustrating the invention, and should not be construed as limiting.

### EXAMPLES

The following definitions are used in the examples:

Hct %: hematocrit, %

RBC: red blood cells

RBCC: red blood cell concentrate

CPD/AS-1 RBCC: CPD collected RBCC supplemented with AS-1 additive solution

CE: cation-exchange

PC: post-column

AMW: after manual wash

AAW: after automated wash

RT: room temperature

ND: no data

Materials

Chemicals:

DOWEX™ 50W×8 resin was obtained from SUPELCO (Catalog # 50x8100). DOWEX 50W×8 resin is a 8% cross-linked polystyrene gel with the following properties: bead size 150-300 micron (50-100 mesh); sulfonic cation-exchange group; swelling in saline: 1.9 mL/g; volume of 1 g of dry resin: 1.2 mL; exchange capacity: 1.7 meq/mL of wet resin; ionic form: H⁺.

Dowex MAC3 was obtained from SUPELCO (catalog # 5025458). Dowex MAC3 is polyacrylic, macroporous, weak cation exchange beads with the following properties: bead size 300-1200 micron (16-50 mesh); carboxyl cation-exchange group; swelling in saline: 6 mL/g; volume of 1 g of dry resin:2.2 mL; exchange capacity: 3.8 meq/mL of wet resin; ionic form: H⁺.

Purolite C115 was obtained from Purolite (Catalog# SR 5011357). Purolite C115 is methacrylic, macroporous weak cation exchange beads with the following properties: bead size 300-1200 micron (16-50 mesh); carboxyl cation-exchange group; swelling in saline: 6 mL/g; volume of 1 g of dry resin:2.1 mL; exchange capacity: 3.5 meq/mL of wet resin; ionic form: H⁺.

Methyl alcohol was obtained from Aldrich (catalog # 27,047-4); sodium hydroxide was obtained from J. T. Baker (catalog # 3722-05). PEN110 is a product of V.I. Technologies, Inc. (Watertown, Mass.).

Solutions:

Saline (0.2% dextrose and 0.9% sodium chloride) was obtained from Baxter Fenwal (code 4B7878); AS-3 additive solution was obtained from MedSep.

Equipment:

The following equipment was used: sterile connecting device (Terumo); heat sealer (Zebra); Automated V215 closed RBC washing system for removal of PEN110 (Haemonetics software version 34.05w, centrifuge bowl capacity 325 mL); Haemofuge (Heraeus); Plasma Extractor (Fenwel); GC-6 KR centrifuge (Beckman); laboratory balances (Mettler), Orbitron Rotator II (Boeke Scientific), Vary-Mix (Thermoline).

Supplies:

The following supplies were used: Flex-Columns 2.5×10, 2.5×20, 2.5×50, 2.5×75 and 1.5×30 cm (Kimble); 2 L glass column 7.6×56 cm (Aldrich); 600 mL transfer packs (Baxter); pH indicator stripes.

Human Blood:

Units of leukodepleted (LEUKOTRAP®-SC RC leukocyte reduction filters) CPD/AS-1 RBCC (≦72 hr old) were purchased from the Tennessee Blood Service (Memphis, Tenn.). All units used in the study were delivered to V.I. Technologies by express mail on ice.

### Example 1

### Removal of Ethyleneimine Oligomer from Red Blood Cell Concentrates Using Strong Cation Exchange Resin

INACTINE™ PEN110 is an ethyleneimine oligomer microbicidal compound used in methods of chemical pathogen reduction in biological compositions such as red blood cell concentrates (RBCC). To inactivate viruses and other pathogens in RBCC, the method generally involves the incubation of RBCC with 0.1% (v/v) PEN110 at 23+2° C. for 24 hrs followed by automated washing of RBC to remove PEN110 to a level below 50 ng/mL. Besides consistent PEN110 removal, automated washing effectively removes pathogenic proteins associated with transfusion reactions (e.g., TRALI associated IgG), as well as extra-cellular and, to some extent, intra-cellular PEN110 degradation products and adducts. However, automated washing generates a substantial amount of liquid waste per RBCC unit and it is time- and space-consuming.

DOWEX 50W×8 is a strong polystyrene-based cation-exchanger widely used for demineralization of water [1] and for numerous separation applications in the biotechnology and pharmaceutical industry [2]. A polystyrene cation-exchanger similar to DOWEX 50W×8 was used to reduce the pre-transfusion potassium ion concentration in aged RBCC units [3].

The purpose of the study was to explore an alternative approach to PEN110 removal from RBCC using cation exchange (CE) resins (e.g., DOWEX™ 50W×8 columns). The PEN110 molecule is positively charged at physiological pH and can bind to negatively charged molecules and surfaces. PEN110 removal using CE columns eliminates the use of special washing equipment and therefore may substantially simplify processes of microbial inactivation. Thus, the following experiments were performed to evaluate the efficiency of PEN110 removal from RBCC using DOWEX 50W×8 columns as an exemplary cation exchange resin, to determine the extent of additional post-column PEN110 removal by manual washing, to assess the effect of the amount of cation resin, dimensions of the column, flow rate and temperature on the extent of microbicidal compounds removal from RBCC, to identify the column parameters and chromatography conditions that can provide post-column or post-manual wash PEN110 removal below 50 ng/mL, and to assess the in vitro quality of RBC purified using DOWEX 50W×8 columns.

Methods

Preparation of DOWEX 50W×8 in Na⁺Form:

830 g (approximately 1 L) of dry DOWEX 50W×8 resin in hydrogen form was suspended in 2 L of deionized water and gently mixed. Supernatant fluid was removed by decanting. The resin was washed again with 2 L of water and loaded on a 7.5×56 cm (2 L) glass column, with a sintered filter. The resin on the column was washed with ˜1.5 L of 1 M NaOH. The pH of the eluate was monitored using pH indicator strips. Washing was continued until the pH of eluate reached 13-14. The resin was then washed with deionized water (˜4 L) until the pH of the eluate decreased to 7. Finally, the resin was washed with 2 L of methanol, transferred to a 1 L thick-walled plastic container, and dried under reduced pressure (6 mm Hg) for 24 hrs at room temperature.

Preparation of DOWEX 50W×8 Columns:

The suspension of DOWEX 50W×8 in saline (2.5 ml/1 g of dry resin) was loaded onto Flex-Columns of different sizes to the final volumes and weights of resin shown in Tables 1-5. The columns filled with wet resin were sterilized with 10-15 kGy at Steris Corporation (Westborough, Mass.). Immediately before use the columns were flushed with 2-4 column volumes of saline.

PEN110 Treatment of RBCC:

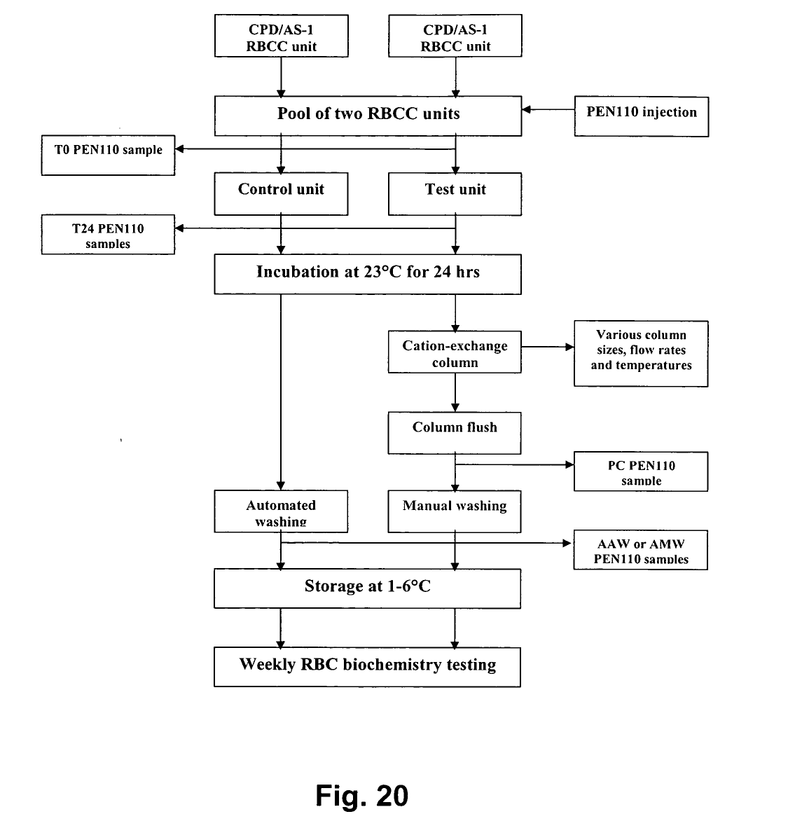

Two to four compatible units of CPD/AS-1 RBCC were pooled together (see Experimental flow chart, FIG. 20) and injected with PEN110 to a final concentration of 0.1% (v/v). The PEN110 was delivered to the pool as a neutral 2% (v/v) 20x stock solution in 0.25 M filter-sterilized NaH₂PO₄. Immediately after PEN110 delivery the RBCC pool was split into two to four identical units in 600 mL transfer packs (Baxter). The units were incubated for 24 hr at 23° C.

Control RBCC Units:

One unit from each set of identical PEN110-treated RBCC units was washed with saline containing 0.2% dextrose and 0.9% sodium chloride (Baxter Fenwal) using the automated Haemonetics V215 RBC washing system (software version 34.05 w, centrifuge bowl capacity 325 mL).

Removal of PEN110 from RBCC Using DOWEX 50W×8 Columns.

The remaining PEN110-treated RBCC units (Test) from each set were passed by gravity through DOWEX 50W×8 columns of different sizes with various average flow rates (1-8 mL/min) at various temperatures (20-30° C.) and collected in 600 mL transfer packs. For better recovery of RBC, the columns were flushed with 140 mL (for 2.5×10, 2.5×20, 2.5×30 and 1.5×30 cm columns) or 250 mL (for 2.5×50 and 2.5×75 cm columns) of saline. The average column flow rate was controlled by clamping the line between the incubation bag and the column. The experiments at different temperatures were conducted in different laboratories with room temperature adjusted to 20±1, 26±1 or 30±1° C.

Manual Wash:

The following steps were performed for additional purification of RBC: 1) RBC were spun down by centrifugation (2500 rpm, 1450 g, 5 min); 2) the supernatants were expressed to the empty transfer packs; 3) saline (300 mL) was sterilely added to the packed RBC; 4) RBCC units were placed on an orbital shaker and equilibrated with saline at 15 rpm for 10 min; 5) Sequence of steps 1), 2), 3), 4), 1) and 2) were repeated; 6) Packed RBC were supplemented with AS-3 to final Hct 60%.

PEN110 Quantitation:

Aliquots of RBCC (˜1 mL) were removed as follows: 1) from PEN110 treated RBCC pool (T0); 2) from each RBCC unit after 24 hr incubation with PEN110 (T24); 3) from column purified units post column purification (PC) and after manual wash (AMW). From Control units the samples were collected from the final product after automated wash (AAW). The samples for PEN110 quantitation were prepared by mixing 250 μL of RBCC with 1 mL of water (dilution 1:5), using the following procedure.

Preparation of Samples for PEN110 Quantitation:

Microcentrifuge tubes (2 mL) were labeled with sample ID, date and initials. 800 μL of deionized water were pipetted into each microcentrifuge tube using a 2000 μL capacity pipette. Using a 250 μL pipette, 200 μL of the RBC concentrate (RBCC) sample was aliquotted into the tip, the tip was wiped and the 200 μL of RBCC was transferred into the sample tube containing the deionized water. The tip was rinsed three times by pipetting up and down to ensure proper delivery.

The sample tubes were capped and the sample was mixed by placing the sample tube on vortex mixer to ensure absence of RBC aggregates in the solution.

The above steps were repeated for each sample tube, replacing the tip each time.

The residual level of PEN110 in RBCC was determined by cation-exchange HPLC coupled with post-column OPA derivatization using the following procedure in RBC lysates, low range. Parallel quantitation of PEN110 in PC, AMW and AAW samples was done by LC/MS.

The HPLC method utilizes cation exchange on a polystyrene divinylbenzene column with benzenesulfonic acid functional groups (Pickering Alkion action exchange column, 4×150 mm, equipped with 3×20 mm guard column; gradient elution using Pickering buffers). Since the PEN110 molecule does not contain a natural chromophore, post-column derivatization is needed for detection. For this assay, post-column modification of the primary amino group with o-phthalaldehyde in the presence of β-mercaptoethanol produced a highly fluorescent product (optimal excitation at 330 nm and maximal emission at 465 nm) that is detected by fluorescence.

In preparation for the quantitation of PEN110 in RBC concentrates (RBCC), a test sample of RBCC was diluted 5× in deionized water to lyse the cells and frozen at −80° C. The aliquot of test sample was transferred into a Microcon YM-3 spin filter and centrifuged prior to loading on the column for analysis. Quantitative recovery of free PEN110 from red cell concentrates and from the sample lysate has been demonstrated by spiking experiments. The concentration of PEN110 in the test sample was measured by linear regression analysis from a standard curve (for example 0.5, 0.7, 1.0, 2.0, 3.0, 6.0 ng/mL) of PEN110 diluted in RBC blank lysate using Beckman 32 Karat software.

Storage:

Column purified and Control RBCC units were stored for 42 days (days of storage were counted from the collection day) at 1-6° C. in AS-3 (Nutricel).

RBC Biochemical Parameters:

Aliquots of 10 mL were removed weekly from each RBCC unit to measure the following parameters, as described below: % RBC hemolysis; ATP (μmole/g Hgb); methemoglobin as % of total hemoglobin; hematocrit (%); red blood cell intra-cellular and extra-cellular potassium level (mEq/10¹² RBC and mEq/L respectively); mean corpuscular volume (MCV, fL); pH; total hemoglobin (g/dL); mean cellular hemoglobin concentration (MCHC, g/dL); osmolality (mOsm); and RBC count (10⁶/μL).

A. Measurement of Mean Corpuscular Volume (MCV) and Red Blood Cell Count:

Samples were analyzed using a Beckman-Coulter ONYX analyzer, either using the “autoloader” function or manually, following the manufacturer's instructions. Beckman-Coulter 4C Plus Cell Controls were used for calibration of the analyzer.

Data Calculation for Mean Corpuscular Volume (MCV) and Red Blood Cell Count:

The red blood cell (RBC) count is the number of erythrocytes measured directly, multiplied by the calibration constant and expressed as follows:

RBC=n×10⁶ cells per μL.

The mean corpuscular volume (MCV) is the average volume of individual erythrocytes derived from the RBC histogram. The analyzer's data processing system multiplies the number of RBCs in each channel by the size of the RBCs in that channel. The products of each channel between 36 fL and 360 fL are added. This sum is divided by the total number of RBCs between 36 fL and 360 fL. The analyzer then multiplies the value by a calibration constant and expresses MCV in femtoliters.

Adjusted Hct=Hct×0.95 MCV=(Adjusted Hct×10)÷RBC

Data Calculation for Hemoglobin (Hgb) Concentration:

The Beckman Coulter ONYX Database Management System converts a ratio that is calculated between transmittance of monochromatic light (525 nm wavelength) through a standard path length of hemoglobin solution and the transmittance of such light in the same way through the reference (diluent) to absorbance. It then converts absorbance to hemoglobin values in g/dL using a calibration factor. Weight (mass) of hemoglobin is determined from the degree of absorbance found through photocurrent transmittance. The Database Management System performs the following calculation:

$$Hemoglobin ( g \text{/} dL ) = Constant \times log10 ( \frac{Reference \% T}{Sample \% T} )$$

where T=% Transmittance

RBCC volumes were determined by dividing the RBCC weights (g) by RBCC densities (d, g/mL). The densities of CPD/AS-1 RBCC were calculated using the formula: d=0.00075×Hct %+1.014. The densities of RBCC supplemented with AS-3 (post automated or manual wash) were calculated using the formula d=0.00071×(Hct %)+1.017 [4]. Spun Hct % were measured according to the following method using a Damon Microhematocrit Reader.

B. Measurement of Hematocrit

Materials

Hema-Trol Controls (Low, Normal, High), J&S Medical, cat. # 84651. Hema-Trol is composed of stabilized human RBC suspended in buffered bacteriostatic and fungistatic fluid.

Calibration Using Hemo-Trol Controls:

Each vial was vigorously mixed by alternating between a vortex mixer and hand shaking (with complete inversion of the vial) to ensure that all of the packed cells have been mixed from the bottom and the sides of the vial. This process was repeated for each level of Hema-Trol control just prior to sampling for measurement.

The Low, Normal and High levels of Hema-Trol were run as a regular sample according to the procedure below. The expected results for the Hema-Trol controls are as follows:

Hema-Trol Control Low 20.0±3.0%

Hema-Trol Control Normal 40.4±3.0%

Hema-Trol Control High 48.0±3.0%

Sample Measurement:

A capillary tube was filled with blood. The tube was wiped, and the end sealed with capillary tube sealer. The capillary tube was spun in a Haemofuge (Baxter) for 5 minutes at 12,000 rpm. The tubes were removed one by one and read immediately using the Microhematocrit Reader (Damon IEC Division 2201) in accordance with the manufacturer's instructions. The tubes were aligned so that the top of the seal plug matched the black line on the plastic capillary holder on the reader. The outer scale was turned until it reached 100%. The inner disk was rotated so that the curved black line matched up with the meniscus of the plasma. because each capillary tube may have been filled to a different level, the 100% value was adjusted for each measurement.

C. Determination of Supernatant Hemoglobin in Red Blood Cells Using Spectrophotometry as a Measure of Hemolysis

When a red blood cell ruptures, Hgb is released as a plasma-free hemoglobin. All forms of hemoglobin in the blood (oxyhemoglobin, reduced hemoglobin, carboxyhemoglobin, and methemoglobin) are quantitatively converted to cyanmethemoglobin upon the addition of Drabkin reagent. Drabkin reagent contains: sodium bicarbonate (NaHCO₃), potassium cyanide (KCN) and potassium ferricyanide (K₃Fe(CN)₆)-Potassium ferricyanide converts hemoglobin iron from the ferrous to the ferric state to form methemoglobin, which combines with potassium cyanide to produce the stable cyanmethemoglobin. The absorption band of cyanmethemoglobin is 540 nm, which is measured by the spectrophotometer.

Calibration (Preparation of a Standard Curve):

A standard curve is generated each time new Drabkin's reagent was prepared. The wavelength of the spectrophotometer was adjusted to 540 nm. The standard curve for total hemoglobin (Total Hgb, g/dL) was generated using the total hemoglobin standard kit (Sigma #525-A) which contains Drabkin's reagent, 30% BRIJ-35 solution, and a lyophilized hemoglobin standard (18 g/dL).

One vial of the Drabkin's reagent was reconstituted with 1100 mL of distilled water and 0.5 mL of the 30% BRIJ-35 solution was added. The Drabkin's solution may be stored at room temperature (18-26° C.) in an amber bottle for up to 6 months; if it appears turbid or cloudy in appearance, a fresh batch must be prepared.

The lyophilized hemoglobin standard was reconstituted with 50 mL of the Drabkin's solution to prepare an 18 g/dL Hemoglobin solution. The following solutions were pipetted to prepare the standard curve:

| | Hemoglobin | Drabkin's | Hemoglobin |
|---|---|---|---|
| Tube No. | Solution | Solution | Concentration |
| 1 | 0.0 mL | 6.0 mL | 0.0 g/dL |
| 2 | 2.0 mL | 4.0 mL | 6.0 g/dL |
| 3 | 4.0 mL | 2.0 mL | 12.0 g/dL |
| 4 | 6.0 mL | 0.0 mL | 18.0 g/dL |

Diluted standards are stable for as long as 6 months when stored tightly capped, in the dark at 4° C.

Tube #1 was placed into the spectrophotometer (Spectronics 20, Genesys) and the absorbance value was set to zero. The absorbance values for tubes 2 through 4 were read and recorded. A standard curve (absorbance value vs. hemoglobin concentration) was plotted.

Using the absorbance values measured for each of the cyanmethemoglobin standards for total hemoglobin, the extraction coefficient (K) was calculated for the three hemoglobin standard solutions (6.0, 12.0 and 18.0 g/dL) using the following formula:

K=Concentration/Absorbance

The average (K₁) was calculated for the three hemoglobin standard solutions (6.0, 12.0 and 18.0 g/dL).

Since the total hemoglobin is measured using a 0.02 mL blood sample diluted with 5.98 mL of cyanmethemoglobin reagent, and the supernatant hemoglobin is made using a 0.3 mL sample of supernatant diluted with 4.7 mL of cyanmethemoglobin reagent, the overall increase observed is 18 fold for the supernatant hemoglobin samples. The following formula is used to calculate the supernatant hemoglobin coefficient (K₂):

$${K}_{2} ( mg \text{/} dL ) = \frac{( {K}_{1} \times 1100 )}{18}$$

Sample Preparation:

15 mL of anti-coagulated blood was placed into a 15 mL plastic test tube. The blood sample was centrifuged in a Beckman Allegra centrifuge set at 2400 rpm, 4 minutes at room temperature, with no brakes. The supernatant was transferred into an Eppendorf tube, using a disposable transfer pipet. The supernatant was centrifuged in the Eppendorf tube in a centrifuge set at 4° C. for 1 min, 3000 rpm. The test was performed without delay, as soon as the samples were ready.

Sample Measurement:

4.7 mL of Drabkin's Solution was dispensed into a glass test tube. A 0.3 mL sample of supernatant fluid was pipetted into the tube; the mixture was vortexed and equilibrated for 2 minutes.

The spectrophotometer was blanked using a glass tube containing 5 mL of Drabkin's solution. The absorbance was adjusted to 0. Absorbance values of the samples was determined. If the value was too high, i.e. >0.6 on the spectrophotometer, an appropriate dilution of the sample was made using the Drabkin's reagent. Referring to the supernatant hemoglobin coefficient (K₂), the hemoglobin concentration was determined, with multiplication by an applicable dilution factor if dilution was performed on a sample:

Sup. Hgb (mg/dL)=Absorbance×K₂×Dilution Factor.

The expected raw data range for the absorbance is 0.01 to 0.4.

D. Quantitative Enzymatic Determination of Intracellular Adenosine-5′-Triphosphate (ATP) in Red Blood Cells

The ATP content of red blood cells is measured by preparing a protein free supernatant (lysing cells) from CPD or CP2D whole blood, which is then corrected later for the hemoglobin concentration. In the described procedure, the enzyme, phosphoglycerate phosphokinase(PGK), is used to catalyze the following reaction:

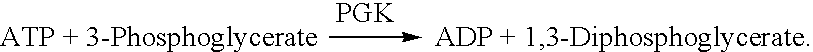

The enzyme glyceraldehyde phosphate dehydrogenase (GAPD) is also present in the reaction mixture to catalyze the following:

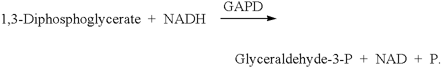

By determining the decrease in absorbance at 340 nm that results when NADH is oxidized to NAD, a measure of the amount of ATP originally present is obtained.

Materials

NADH Vial (Sigma, cat. # 340-13); PGA Buffered Solution (Sigma, cat. # 366-1); 12% TCA (Sigma, cat. # 366-12); GAPD/PGK Enzyme Mixture (Sigma, cat. # 366-2).

Sample Preparation (Protein-Free Supernatant):

1.0 mL 12% TCA and 1.0 mL RBC blood were pipetted into a centrifuge tube. The tube was inverted and shaken, then the mixture was allowed to stand approximately 5 minutes in ice bath. The mixture was centrifuged at about 2500 rpm, 5 minutes at 23° C., to obtain clear supernatant. The supernatant was transferred to an eppendorf tube and centrifuged for 5 min, 5000 rpm at 4° C.

Sample Measurement:

Blood samples were processed immediately, or if not a note was made regarding how long the sample was stored and at what temperature. The ATP level tends to decrease with storage. Samples were kept on ice prior to assay.

The spectrophotometer (Spectronics 20, Genesys) was set to a the wavelength of 340 nm. A blank glass tube containing deionized water (Milli-Q, Millipore Corporation) was inserted into the spectrophotometer and the “ABS” button was pressed to set the spectrophotometer to zero.

The following solutions were pipetted into a 0.3 mg NADH Vial, in the order indicated: 1.0 mL PGA Buffered Solution; then 1.5 mL water; then 0.5 mL supernatant. The contents of the tube were mixed using a vortex mixer for approximately five seconds to dissolve the NADH. The entire contents were decanted into a glass tube, and the Initial Absorbance (A₁) vs. water as reference was read and recorded at 340 nm. An Initial Absorbance value lower than 0.6 may indicate NADH decomposition. If this occurred, the procedure was repeated with a new 0.3 mg NADH vial.

0.04 mL of the GAPD/PGK Enzyme Mixture was pipetted into the glass tube. The mixture was mixed and incubated for 10 minutes at room temperature. The absorbance vs. water as a reference was read and recorded at 340 nm. This was recorded as the Final Absorbance (A₂).

Calculation of Results:

The ATP concentration is calculated as follows:

$$Hemoglobin ATP ( µmol \text{/} g Hb ) = \frac{Δ A \times 195}{Blood Hemoglobin ( g \text{/} dL )}$$

Where:ΔA=A₁-A₂

$$The factor 195 is derived as follows \text{:} \frac{3.04 \times 100}{6.22 \times 0.25} = 195$$

Where: 3.04=Volume of liquid in glass tube

100=Conversion of concentration per mL to concentration per dL

6.22=Millimolar absorptivity of NADH at 340 nm

0.25=Sample volume

The normal expected range for ATP in RBCC is 3.65-4.45 μmol/g Hb. Washed and stored RBCC will show some ATP loss over the course of 42 day storage. ATP concentration in those cells range from 1.00-3.00 μmol/g Hb.

E. Measurement of Methemoglobin in Blood with the IL 682 Co-Oximeter System

Methemoglobin is a particular type of hemoglobin that is altered so that it is useless for carrying oxygen and delivering it to tissues throughout the human body. Since hemoglobin is the key carrier of oxygen in the blood, its wholesale replacement by methemoglobin can cause cyanosis (a slate gray-blueness) due to lack of oxygen. A small amount of methemoglobin is normally present in blood, but the conversion of a larger fraction of hemoglobin into methemoglobin, which does not function reversibly as an oxygen carrier, results in severe problems.

Methemoglobin is a transformation product of normal hemoglobin (oxyhemoglobin) and is produced by the oxidation of the normal ferrous iron contained in the heme part of hemoglobin to ferric iron which, in firm union with water, is chemically useless for respiration.

Materials and Equipment

All from Instrumentation Laboratory (Lexington, Mass.): Co-Oximeter (IL-682); Diluent (cat # 03311900); Zeroing Solution (cat # 03310700); Cleaning Agent (cat # 09832700); Calibration Dye Reference Solution (cat # 03315050).

Procedure

The procedure was conducted in accordance with the manufacturer's instructions (Instrumentation Laboratory IL-682 Co-Oximeter Operator's Manual). Briefly, a blood sample is aspirated into the instrument, mixed with diluent, hemolysed and brought to a constant temperature in cuvette. Monochromatic light at four specific wavelengths passes through the cuvette to a photo-detector, whose output is used to generate absorbances. The results are reported in Table 8.

F. Measurement of Hematocrit During Storage of Red Blood Cell Concentrates

Measurement of Hct % was performed as described above. The results are reported in Table 9.

G. Measurement of Intracellular and Extracellular Sodium and Potassium in Red Blood Cells

Intracellular and extracellular sodium and potassium in red blood cells was measured using a flame photometer (Instrumentation Labs, IL943) in accordance with the manufacturer's instructions.

Materials

Instrument Grade Propane (Curtin Matheson Scientific, cat # 282-747)

Cesium Diluent Solution (Instrumentation Laboratories, cat # 33310-10)

Rinse Solution (Instrumentation Laboratories, cat # 33110)

Calibration Standard (New England Reagent Labs, cat # 2210)

Calibration Standard (100 mEq/L; New England Reagent Labs, cat # 2214)

Sample Preparation (RBCC Lysates):

Sufficient Eppendorf tubes for the number of samples were prepared by adding 500 μL of Milli-Q water to each. 500 μL of the RBC sample was added to the tubes and vortexed. The samples were frozen at −80° C., for 1 hour. The samples then were centrifuged for 10 minutes, at 13,000 rpm. The supernatant was used as a sample for measurement of intracellular Na⁺ and K⁺ following the manufacturer's instructions.

Results Calculation:

Results for both intracellular and extracellular sodium and potassium were reported in mEq/L. To calculate the intracellular level of Na⁺ and K⁺ in mEq/10-12 RBC, the following formula was used:

$$\frac{Measured RBC Value - ( Plasma Value ) \times ( 0.03 )}{0.97} \times \frac{MVC}{1000}$$

The above formula corrects for plasma trapping. The results for extracellular and intracellular potassium levels are shown in Tables 10 and 11.

H. Measurement of pH for Red Blood Cell Concentrate

pH of RBCCs were measured using the Beckman φ300 pH meter in accordance with the manufacturer's instructions.

Sample Preparation:

0.5 mL of RBCC was placed in a 2 mL Eppendorf tube. After opening the ventilation hole on the pH electrode, the electrode was immersed in the sample and the pH value was recorded. The electrode was rinsed with 50% bleach, followed by a Milli-Q water rinse.

I. Measurement of Supernatant Osmolality Using Fiske 2400 Multi-Sample Osmometer

RBCC osmolality was measured using the Fiske Osmometer 2400 in accordance with the manufacturer's instructions. This procedure is based on the measurement of supernatant osmolality in plasma or supernatant from deglycerolized red blood cells using freezing point depression osmometry.

Sample Preparation:

Samples of RBC concentrate blood were put into plastic tubes (Falcon #2059) and centrifuged in a centrifuge (e.g., Sorval GLC-2B) at 2200 RPM for 10 minutes. Upon completion of centrifugation, the resulting supernatant sample was transferred using a disposable transfer pipet into a smaller plastic tube. The samples prepared as described above may be tightly capped and stored at 4° C. for up to 24 hours prior to assay.

An experimental flow chart is provided as FIG. 20.

Results

Effect of the Amount of DOWEX 50W×8 Resin on PEN110 Removal from RBCC.

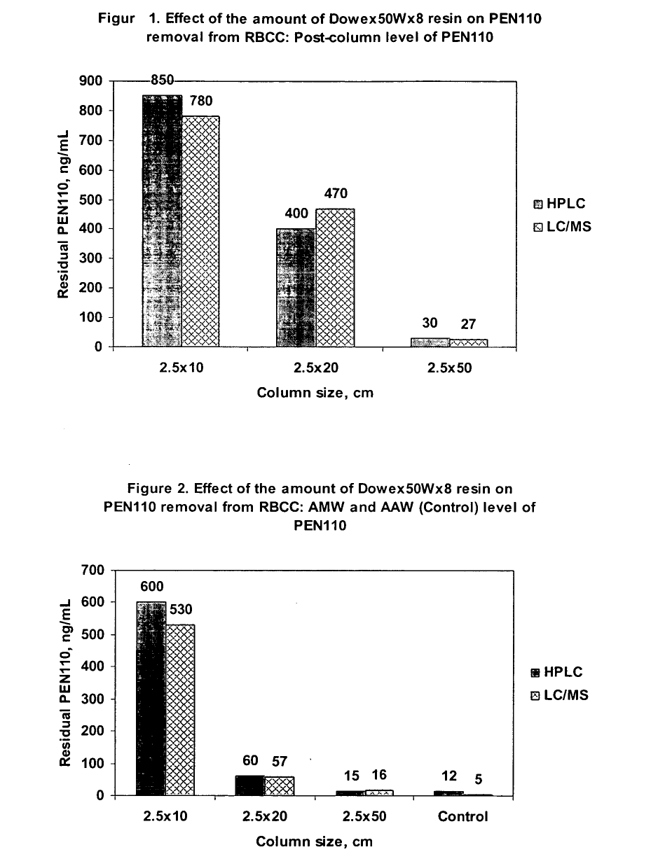

Three identical units of PEN110-treated RBCC were purified using there different columns (2.5×10, 2.5×20 and 2.5×50 cm) filled with different amounts of DOWEX 50W×8 resin following the purification steps depicted in the experimental flow chart (FIG. 20). Experimental conditions and the results of PEN110 quantitation by two analytical methods are shown in Table 1 and FIGS. 1 and 2.

TABLE 1
Effect of the amount of DOWEX 50WX8 resin on PEN110 removal from
RBCC: Parameters of DOWEX 50WX8 columns tested, experimental conditions and
PEN110 quantitation results.

| | | | | | | | | PEN110 concentration | | | |
|---|---|---|---|---|---|---|---|---|---|---|---|
| | Experimental parameters | | | | | | | | | | AAW or |
| | | Volume | Weight | Average | | | | | | | AMW |
| | Column | of wet | of dry | flow | | RBCC | | T0 | T24 | PC HPLC | HPLC |
| Experiment | size, | resin, | resin, | rate, | RT, | volume, | Hct, | HPLC, | HPLC, | (LC/MS), | (LC/MS), |
| Unit | cm | mL | g | mL/min | ° C. | mL | % | μg/mL | μg/mL | ng/mL | ng/mL |
| 1-Test-1 | 2.5 × 10 | 49 | 25 | 1-2 | 26 ± 1 | 336 | 53 | 946 | 600 | 850 (780) | 600 (530) |
| 1-Test-2 | 2.5 × 20 | 98 | 50 | 1-2 | | 337 | | | | 400 (470) | 60 (57) |
| 1-Test-3 | 2.5 × 50 | 246 | 130 | 1-2 | | 336 | | | | 30 (27) | 15 (16) |
| 1-Control | NA | NA | NA | NA | | 336 | | | | NA | 12 (5) |

Effect of Flow Rate on PEN110 Removal from RBCC Using DOWEX 50W×8 Columns.

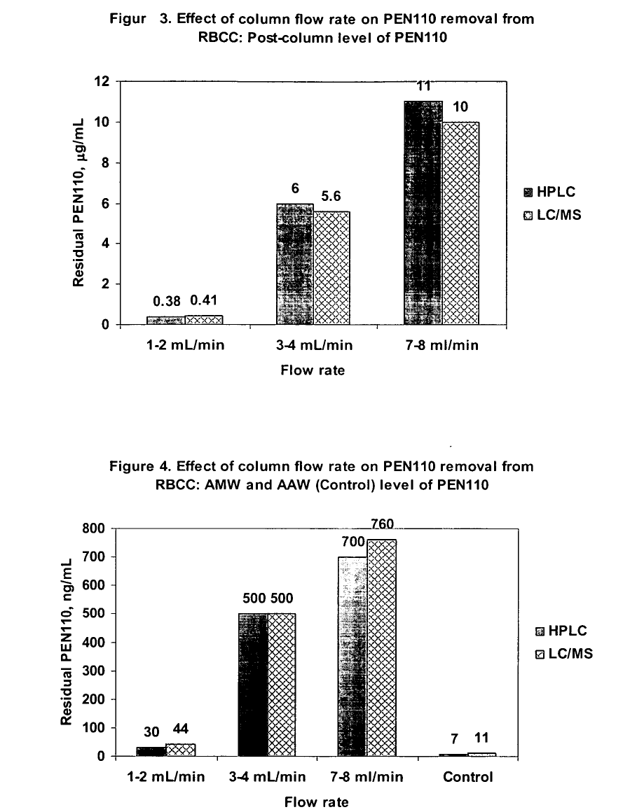

Three identical units of PEN110-treated RBCC were purified using 2.5×30 cm DOWEX 50W×8 columns following the purification steps depicted on the experimental flow chart (FIG. 20). RBCC were passed through the columns with different average flow rates: 1-2 ml/min, 3-4 ml/min and 6-8 ml/min. Experimental conditions and PEN110 quantitation results by two analytical methods are shown in Table 2 and FIGS. 3 and 4.

TABLE 2
Effect of RBCC flow rate of on PEN110 removal: Parameters of DOWEX
50WX8 columns tested, experimental conditions and PEN110 quantitation results.

| | | | | | | | | PEN110 concentration | | | |
|---|---|---|---|---|---|---|---|---|---|---|---|
| | Experimental parameters | | | | | | | | | | AAW or |
| | | Volume | Weight | Average | | | | | | PC | AMW |
| | Column | of wet | of dry | flow | | RBCC | | T0 | T24 | HPLC | HPLC |
| Experiment | size, | resin, | resin, | rate, | RT, | volume, | Hct, | HPLC, | HPLC, | (LC/MS), | (LC/MS), |
| Unit | cm | mL | g | mL/min | ° C. | mL | % | μg/mL | μg/mL | ng/mL | ng/mL |
| 2-Test-1 | 2.5 × 30 | 147 | 75 | 1-2 | 26 ± 1 | 334 | 58 | 854 | 476 | 380 (410) | 30 (44) |
| 2-Test-2 | 2.5 × 30 | 147 | 75 | 3-4 | | 336 | | | | 6000 (5600) | 500 (500) |
| 2-Test-3 | 2.5 × 30 | 147 | 75 | 7-8 | | 335 | | | | 11000 (10000) | 700 (760) |
| 2-Control | NA | NA | NA | NA | | 334 | | | | NA | 7 (11) |

Effect of Temperature on PEN110 Removal from RBCC Using DOWEX 50W×8 Columns.

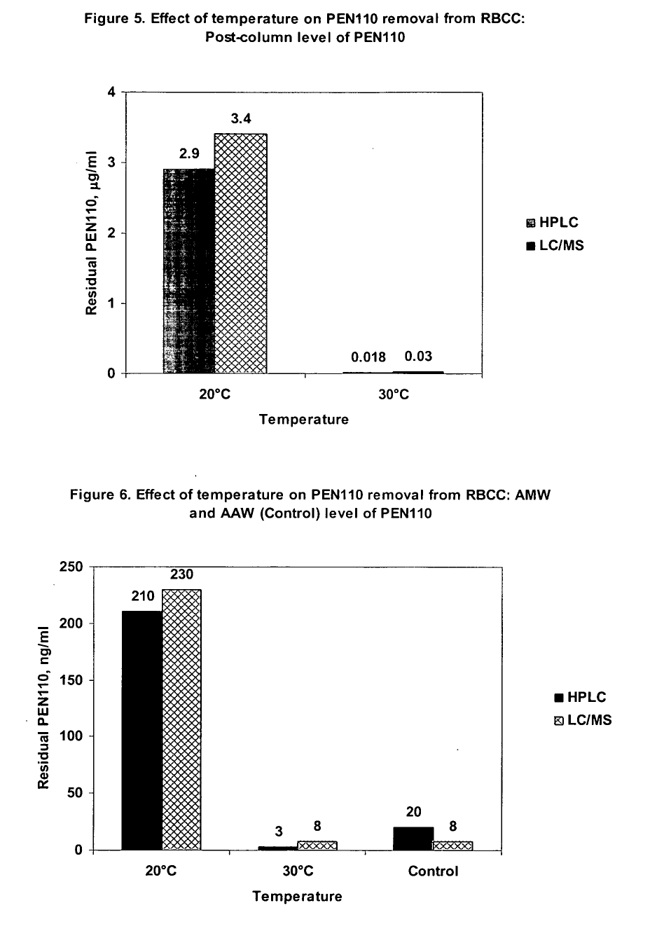

Two identical units of PEN110 treated RBCC were purified using 2.5×50 cm DOWEX 50W×8 columns at 20±1° C. and 30±1° C. following the purification steps depicted on the experimental flow chart (FIG. 20). Experimental conditions and the results of PEN110 quantitation by two analytical methods are shown in Table 3 and FIGS. 5 and 6.

TABLE 3
Effect of temperature on PEN110 removal from RBCC: Parameters of
DOWEX 50WX8 columns tested, experimental conditions and PEN110 quantitation
results.

| | | | | | | | | PEN110 concentration | | | |
|---|---|---|---|---|---|---|---|---|---|---|---|
| | Experimental parameters | | | | | | | | | | AAW or |
| | | Volume | Weight | Average | | | | | | PC | AMW |
| | Column | of wet | of dry | flow | | RBCC | | T0 | T24 | HPLC | HPLC |
| Experiment | size, | resin, | resin, | rate, | RT, | volume, | Hct, | HPLC, | HPLC, | (LC/MS), | (LC/MS), |
| Unit | cm | mL | g | mL/min | ° C. | mL | % | μg/mL | μg/mL | ng/mL | ng/mL |
| 3-Test-1 | 2.5 × 50 | 246 | 130 | 4-5 | 20 ± 1 | 334 | 58 | 921 | 507 | 2900 (3400) | 210 (230) |
| 3-Test-2 | 2.5 × 50 | 246 | 130 | 4-5 | 30 ± 1 | 336 | | | | 18 (30) | 3 (8) |
| 3-Control | NA | NA | NA | NA | 30 ± 1 | 334 | | | | NA | 20 (8) |

Effect of Column Configuration on PEN110 Removal from RBCC Using DOWEX 50W×8 Columns.

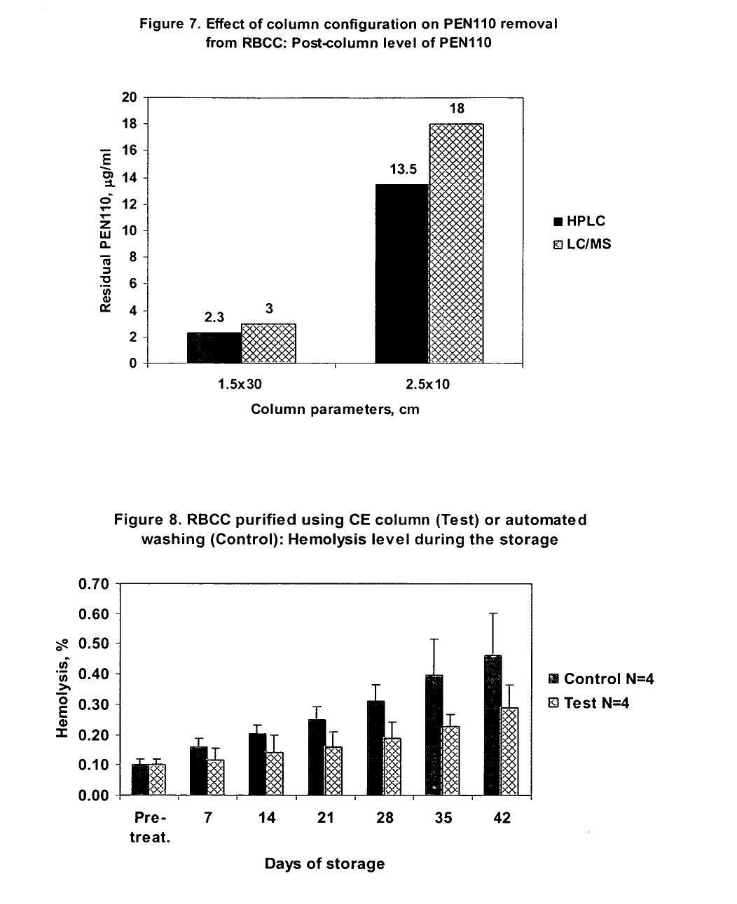

Two identical units of PEN110-treated RBCC were purified using 1.5×30 cm and 2.5×10 cm DOWEX 50W×8 columns packed with 25 g of DOWEX 50W×8 following the purification steps depicted on the experimental flow chart(FIG. 20). Experimental conditions and the results of PEN110 quantitation by two analytical methods are shown in Table 4 and FIG. 7.

TABLE 4
Effect of column configuration on PEN110 removal from RBCC: Parameters
of DOWEX 50WX8 columns tested, experimental conditions and PEN110 quantitation
results.

| | | | | | | | | PEN110 concentration | | | |
|---|---|---|---|---|---|---|---|---|---|---|---|
| | Experimental parameters | | | | | | | | | | AAW or |
| | | Volume | Weight | Average | | | | | | PC | AMW |
| | Column | of wet | of dry | flow | | RBCC | | T0 | T24 | HPLC | HPLC |
| Experiment | size, | resin, | resin, | rate, | RT, | volume, | Hct, | HPLC, | HPLC, | (LC/MS), | (LC/MS), |
| Unit | cm | mL | g | mL/min | ° C. | mL | % | μg/mL | μg/mL | ng/mL | ng/mL |
| 4-Test-1 | 1.5 × 30 | 50 | 25 | 2 | 27 ± 1 | 336 | 57 | 882 | NA | 2300 (3000) | N/A |
| 4-Test-2 | 2.5 × 10 | 50 | 25 | 2 | 27 ± 1 | 336 | | | | 14000 (18000) | N/A |
| 4-Control | NA | NA | NA | NA | 27 ± 1 | 340 | | | | NA | 10 (14) |

PEN110 Removal from RBCC Using 2.5×75 cm DOWEX 50W×8 Columns.

Two pairs of identical PEN110-treated RBCC units were prepared. One unit from each pair was purified using 2.5×75 cm DOWEX 50W×8 columns at 27° C. following the purification steps depicted on the experimental flow chart (FIG. 20). Another unit from each pair was washed using the automated Haemonetics V215 RBC washing system. Experimental conditions and the results of PEN110 quantitation by two analytical methods for two pairs of RBCC units are shown in Table 5.

TABLE 5
PEN110 removal from RBCC using 2.5 × 75 cm DOWEX 50WX8 columns:
Parameters of columns tested, experimental conditions and PEN110 quantitation
results.

| | | | | | | | | PEN110 concentration | | | |
|---|---|---|---|---|---|---|---|---|---|---|---|
| | Experimental parameters | | | | | | | | | | AAW or |
| | | Volume | Weight | Average | | | | | | PC | AMW |
| | Column | of wet | of dry | flow | | RBCC | | T0 | T24 | HPLC | HPLC |
| Experiment | size, | resin, | resin, | rate, | RT, | volume, | Hct, | HPLC, | HPLC, | (LC/MS), | (LC/MS), |
| Unit | cm | mL | g | mL/min | ° C. | mL | % | μg/mL | μg/mL | ng/mL | ng/mL |
| 6-Test-1 | 2.5 × 75 | 344 | 180 | 7-8 | 27 ± 1 | 337 | 57 | 882 | ND | 47 (63) | 19 (17) |
| 6-Control | NA | NA | NA | NA | | 340 | | | | NA | 10 (17) |
| 7-Test-1 | 2.5 × 75 | 344 | 180 | 7-8 | | 336 | 58 | 985 | 559 | 26 (33) | 8 (5) |
| 7-Control | NA | NA | NA | NA | | 335 | | | | NA | 5 (8) |

In vitro Quality of PEN110-Treated RBC Purified Using DOWEX 50W×8 Columns.

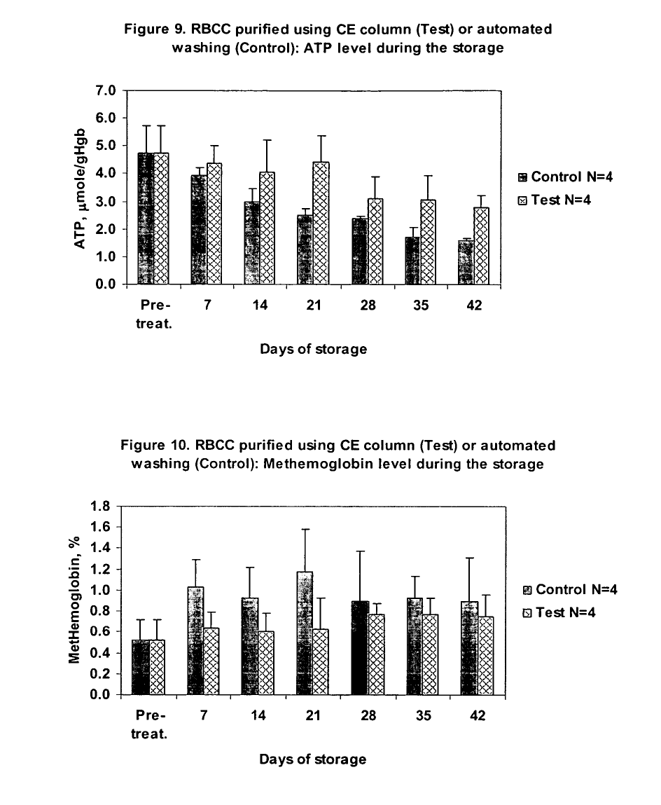

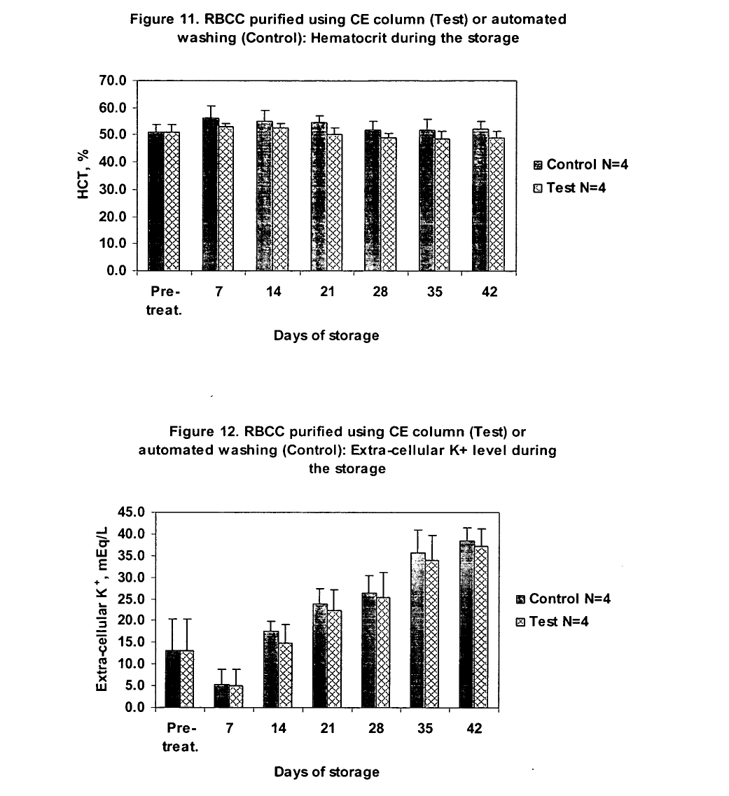

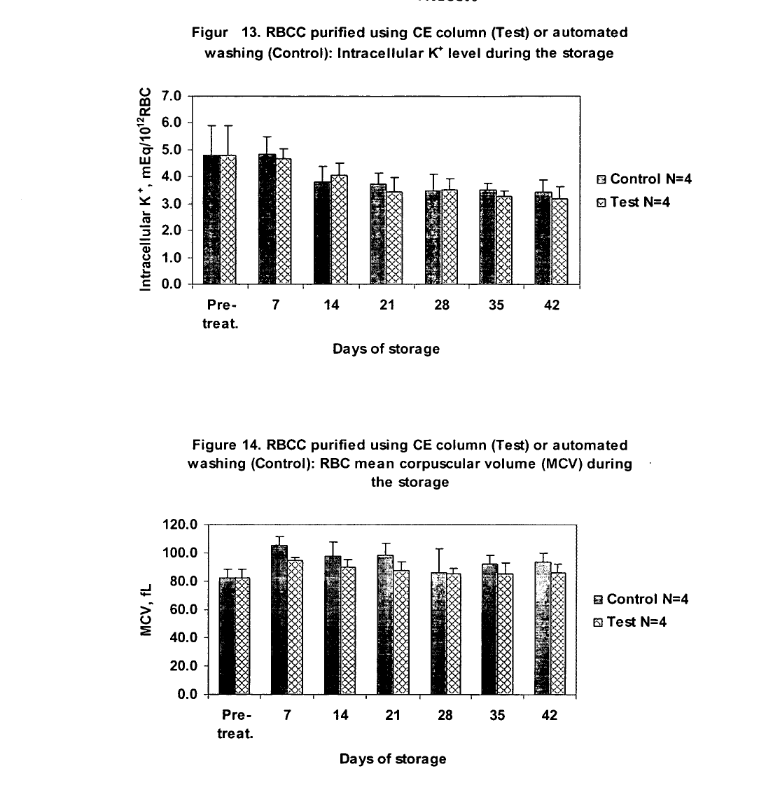

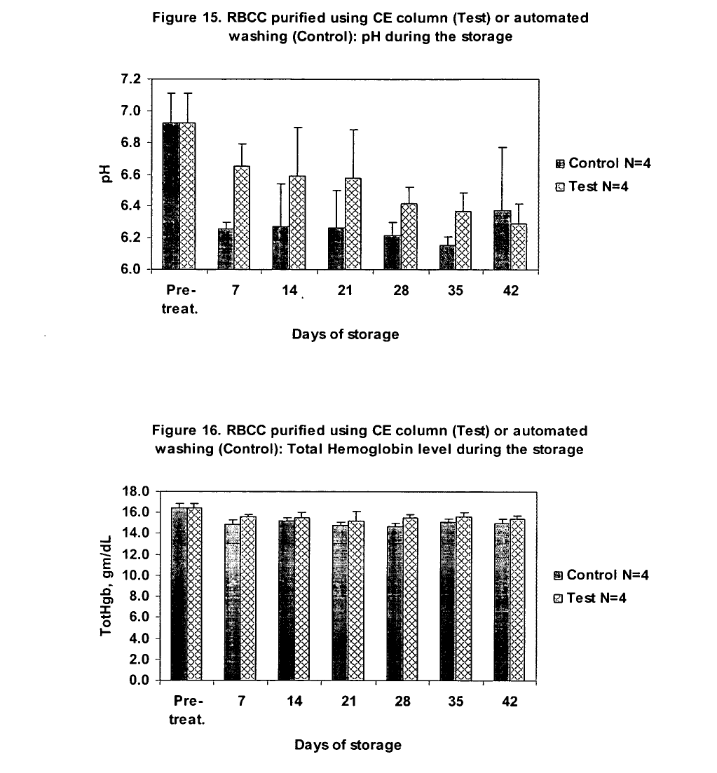

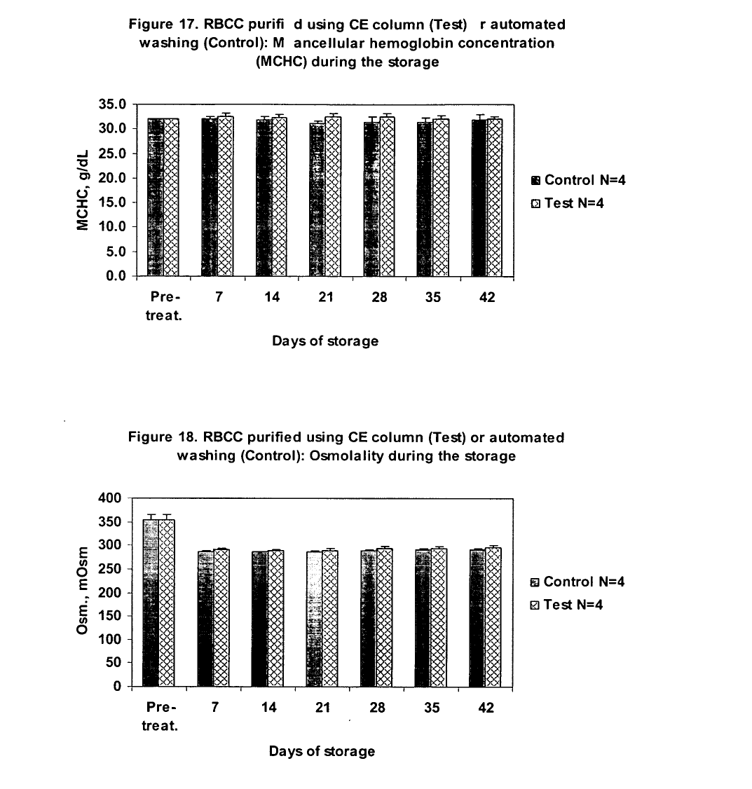

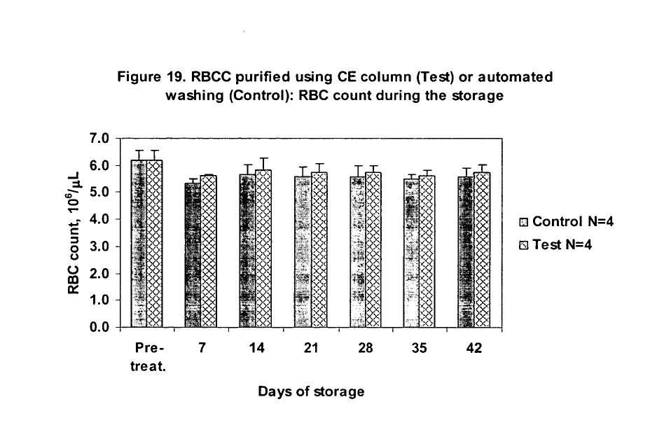

A standard panel of twelve RBC biochemical parameters was measured weekly during the 42 day storage period for four pairs of identical RBCC units treated with PEN110 and purified using DOWEX 50W×8 columns (Test) or automated washing (Control). Study results for the units from thee Test set (1-Test-3, 2-Test-1, 4-Test-2 and 5-Test-1) and the Control set (1-Control, 2-Control, 4-Control and 5-Control) are shown in Tables 6-17 and FIGS. 8-19.

TABLE 6
% Hemolysis during the storage of RBCC at 1-6° C.

| | Hemolysis, % | | | | | | |
|---|---|---|---|---|---|---|---|
| Experiment- | Pre- | | | | | 35 | 42 |
| RBC unit | treat. | 7 days | 14 days | 21 days | 28 days | days | days |
| 1-Control | 0.08 | 0.17 | 0.22 | 0.29 | 0.37 | 0.55 | 0.66 |
| 2-Control | 0.10 | 0.13 | 0.16 | 0.22 | 0.28 | 0.30 | 0.43 |
| 4-Control | 0.12 | ND | 0.21 | 0.28 | 0.34 | 0.43 | 0.43 |
| 5-Control | 0.11 | 0.18 | 0.22 | 0.21 | 0.25 | 0.31 | 0.33 |
| Average | 0.10 | 0.16 | 0.20 | 0.25 | 0.31 | 0.40 | 0.46 |
| SD | 0.02 | 0.03 | 0.03 | 0.04 | 0.05 | 0.12 | 0.14 |
| 1-Test-3 | 0.08 | 0.10 | 0.16 | 0.22 | 0.25 | 0.28 | 0.39 |
| 2-Test-1 | 0.10 | 0.09 | 0.12 | 0.16 | 0.21 | 0.21 | 0.30 |
| 4-Test-2 | 0.12 | ND | 0.08 | 0.10 | 0.13 | 0.19 | 0.21 |
| 5-Test-1 | 0.11 | 0.16 | 0.21 | 0.16 | 0.16 | 0.23 | 0.26 |
| Average | 0.10 | 0.12 | 0.14 | 0.16 | 0.19 | 0.23 | 0.29 |
| SD | 0.02 | 0.04 | 0.06 | 0.05 | 0.05 | 0.04 | 0.08 |

TABLE 7
ATP (μmole/g Hgb) in RBC during the storage of RBCC at 1-6° C.

| | ATP, μmole/g Hgb | | | | | | |
|---|---|---|---|---|---|---|---|
| Experiment- | Pre- | | | | | 35 | 42 |
| RBC unit | treat. | 7 days | 14 days | 21 days | 28 days | days | days |
| 1-Control | 3.7 | 3.7 | 3.6 | 2.4 | 2.4 | 1.8 | 1.7 |
| 2-Control | 5.5 | 4.3 | 3.1 | 2.5 | 2.3 | 1.8 | 1.7 |
| 4-Control | 4.2 | ND | 2.7 | 2.4 | 2.5 | 1.2 | 1.5 |
| 5-Control | 5.6 | 3.8 | 2.6 | 2.9 | 2.3 | 2.1 | 1.6 |
| Average | 4.8 | 3.9 | 3.0 | 2.5 | 2.4 | 1.7 | 1.6 |
| SD | 1.0 | 0.3 | 0.5 | 0.2 | 0.1 | 0.4 | 0.1 |
| 1-Test-3 | 3.7 | 4.0 | 3.3 | 3.3 | 3.3 | 3.1 | 3.0 |
| 2-Test-1 | 5.5 | 5.1 | 5.0 | 4.9 | 3.2 | 3.0 | 2.3 |
| 4-Test-2 | 4.2 | ND | 5.2 | 4.1 | 2.0 | 2.0 | 2.6 |
| 5-Test-1 | 5.6 | 4.0 | 2.9 | 5.5 | 3.9 | 4.1 | 3.2 |
| Average | 4.8 | 4.4 | 4.1 | 4.4 | 3.1 | 3.1 | 2.8 |
| SD | 1.0 | 0.7 | 1.2 | 1.0 | 0.8 | 0.9 | 0.4 |

TABLE 8
% MetHemoglobin formation during the storage of RBCC at 1-6° C.

| | MetHemoglobin, % | | | | | | |
|---|---|---|---|---|---|---|---|
| Experiment- | Pre- | | | | | 35 | 42 |
| RBC unit | treat. | 7 days | 14 days | 21 days | 28 days | days | days |
| 1-Control | 0.5 | 1.0 | 0.9 | 1.4 | 0.6 | 0.8 | 0.6 |
| 2-Control | 0.4 | 1.3 | 1.3 | 1.6 | 1.6 | 1.1 | 0.8 |
| 4-Control | 0.8 | ND | 0.9 | 1.0 | 0.7 | 1.1 | 1.5 |
| 5-Control | 0.4 | 0.8 | 0.6 | 0.7 | 0.7 | 0.7 | 0.7 |
| Average | 0.5 | 1.0 | 0.9 | 1.2 | 0.9 | 0.9 | 0.9 |
| SD | 0.2 | 0.3 | 0.3 | 0.4 | 0.5 | 0.2 | 0.4 |
| 1-Test-3 | 0.5 | 0.6 | 0.4 | 0.2 | 0.7 | 0.7 | 0.5 |
| 2-Test-1 | 0.4 | 0.5 | 0.5 | 0.7 | 0.9 | 1.0 | 0.8 |
| 4-Test-2 | 0.8 | ND | 0.7 | 0.9 | 0.7 | 0.7 | 1.0 |
| 5-Test-1 | 0.4 | 0.8 | 0.8 | 0.7 | 0.8 | 0.7 | 0.7 |
| Average | 0.5 | 0.6 | 0.6 | 0.6 | 0.8 | 0.8 | 0.8 |
| SD | 0.2 | 0.2 | 0.2 | 0.3 | 0.1 | 0.2 | 0.2 |

TABLE 9
Hematocrit (HCT, %) of RBCC during the storage of RBCC at 1-6° C.

| | HCT, % | | | | | | |
|---|---|---|---|---|---|---|---|
| Experiment- | Pre- | | | | | 35 | 42 |
| RBC unit | treat. | 7 days | 14 days | 21 days | 28 days | days | days |
| 1-Control | 47 | 56 | 55 | 53 | 51 | 49 | 48 |
| 2-Control | 51 | 61 | 60 | 58 | 56 | 55 | 53 |
| 4-Control | 51 | ND | 55 | 53 | 51 | 55 | 55 |
| 5-Control | 54 | 52 | 50 | 54 | 48 | 48 | 52 |
| Average | 51 | 56 | 55 | 55 | 52 | 52 | 52 |
| SD | 3 | 4 | 4 | 2 | 3 | 4 | 3 |
| 1-Test-3 | 47 | 53 | 52 | 48 | 48 | 47 | 46 |
| 2-Test-1 | 51 | 54 | 53 | 52 | 50 | 51 | 50 |
| 4-Test-2 | 51 | ND | 54 | 52 | 50 | 50 | 49 |
| 5-Test-1 | 54 | 52 | 50 | 48 | 48 | 47 | 51 |
| Average | 51 | 53 | 52 | 50 | 49 | 49 | 49 |
| SD | 3 | 1 | 2 | 2 | 2 | 2 | 2 |

TABLE 10
Extracellular K⁺ in RBCC during the storage of RBCC at 1-6° C.

| | Extracellular K⁺, mEg/L | | | | | | |
|---|---|---|---|---|---|---|---|
| Experiment- | Pre- | | | | | 35 | 42 |
| RBC unit | treat. | 7 days | 14 days | 21 days | 28 days | days | days |
| 1-Control | 10.1 | 5.0 | 20.3 | 28.5 | 26.5 | 43.5 | 42.6 |
| 2-Control | 18.4 | 2.0 | 18.4 | 21.1 | 20.7 | 31.4 | 36.9 |
| 4-Control | 19.6 | ND | 15.0 | 21.9 | 28.9 | 34.4 | 38.4 |
| 5-Control | 4.5 | 9.0 | 16.5 | 24.4 | 29.5 | 33.0 | 35.5 |
| Average | 13.2 | 5.3 | 17.6 | 24.0 | 26.4 | 35.6 | 38.4 |
| SD | 7.1 | 3.5 | 2.3 | 3.3 | 4.0 | 5.4 | 3.1 |
| 1-Test-3 | 10.1 | 5.0 | 19.6 | 27.0 | 27.2 | 41.3 | 42.0 |
| 2-Test-1 | 18.4 | 1.0 | 9.8 | 16.9 | 17.2 | 27.3 | 32.3 |
| 4-Test-2 | 19.6 | ND | 12.5 | 20.1 | 25.5 | 31.2 | 35.9 |
| 5-Test-1 | 4.5 | 8.9 | 17.2 | 25.4 | 31.3 | 35.5 | 38.7 |
| Average | 13.2 | 5.0 | 14.8 | 22.4 | 25.3 | 33.8 | 37.2 |
| SD | 7.1 | 4.0 | 4.4 | 4.7 | 5.9 | 6.0 | 4.1 |

TABLE 11
Intracellular K⁺ in RBCC during the storage of RBCC at 1-6° C.

| | Intracellular K⁺, mEq/10¹² RBC | | | | | | |
|---|---|---|---|---|---|---|---|
| Experiment- | | | | 21 | 28 | 35 | 42 |
| RBC unit | Pre-treat. | 7 days | 14 days | days | days | days | days |
| 1-Control | 5.1 | 5.1 | 4.2 | 4.2 | 4.1 | 3.3 | 3.2 |
| 2-Control | 3.1 | 5.3 | 4.4 | 3.9 | 5.7 | 3.8 | 3.0 |
| 4-Control | 5.3 | ND | 3.4 | 3.3 | 2.7 | ND | 3.6 |
| 5-Control | 5.6 | 4.1 | 3.3 | 3.5 | 3.5 | 3.5 | 4.0 |
| Average | 4.8 | 4.8 | 3.8 | 3.7 | 4.0 | 3.5 | 3.5 |
| SD | 1.1 | 0.6 | 0.6 | 0.4 | 1.3 | 0.3 | 0.4 |
| 1-Test-3 | 5.1 | 4.4 | 4.1 | 3.8 | 3.9 | 3.0 | 3.4 |
| 2-Test-1 | 3.1 | 4.5 | 4.5 | 3.9 | 3.7 | 3.4 | 2.6 |
| 4-Test-2 | 5.3 | ND | 4.2 | 2.8 | 3.0 | ND | 3.2 |
| 5-Test-1 | 5.6 | 5.1 | 3.4 | 3.3 | 3.5 | 3.4 | 3.6 |
| Average | 4.8 | 4.7 | 4.1 | 3.5 | 3.5 | 3.3 | 3.2 |
| SD | 1.1 | 0.4 | 0.5 | 0.5 | 0.4 | 0.2 | 0.4 |

TABLE 12
Mean Corpuscular Volume (MCV) of RBC
during the storage at 1-6° C.

| | MCV, fL | | | | | | |
|---|---|---|---|---|---|---|---|
| Experiment- | | | | 21 | 28 | 35 | 42 |
| RBC unit | Pre-treat. | 7 days | 14 days | days | days | days | days |
| 1-Control | 78.1 | 103.6 | 100.4 | 97.3 | 62.1 | 87.7 | 87.1 |
| 2-Control | 82.9 | 112.0 | 111.0 | 105.9 | 101.4 | 99.6 | 95.9 |
| 4-Control | 76.6 | ND | 89.2 | 87.4 | 84.2 | ND | 91.5 |
| 5-Control | 91.2 | 101.5 | 89.9 | 103.1 | 95.6 | 89.5 | 101.5 |
| Average | 82.2 | 105.7 | 97.6 | 98.4 | 85.8 | 92.3 | 94.0 |
| SD | 6.6 | 5.6 | 10.3 | 8.2 | 17.4 | 6.4 | 6.2 |
| 1-Test-3 | 78.1 | 93.7 | 89.2 | 85.9 | 83.5 | 79.8 | 80.9 |
| 2-Test-1 | 82.9 | 97.4 | 97.4 | 96.6 | 91.0 | 93.8 | 89.6 |
| 4-Test-2 | 76.6 | ND | 84.1 | 84.5 | 82.7 | ND | 80.3 |
| 5-Test-1 | 91.2 | 92.8 | 89.6 | 85.3 | 84.5 | 83.0 | 92.9 |
| Average | 82.2 | 94.6 | 90.1 | 88.1 | 85.4 | 85.5 | 85.9 |
| SD | 6.6 | 2.4 | 5.5 | 5.7 | 3.8 | 7.3 | 6.3 |

TABLE 13
pH of RBCC during the storage at 1-6° C.

| | pH | | | | | | |
|---|---|---|---|---|---|---|---|
| Experiment- | | | | 21 | 28 | 35 | 42 |
| RBC unit | Pre-treat. | 7 days | 14 days | days | days | days | days |
| 1-Control | 7.2 | 6.3 | 6.7 | 6.6 | 6.2 | 6.2 | 6.2 |
| 2-Control | 6.9 | 6.2 | 6.1 | 6.2 | 6.1 | 6.1 | 6.2 |
| 4-Control | 6.8 | ND | 6.1 | 6.2 | 6.3 | 6.1 | 6.1 |
| 5-Control | 6.9 | 6.3 | 6.1 | 6.1 | 6.2 | 6.2 | 7.0 |
| Average | 6.9 | 6.3 | 6.3 | 6.3 | 6.2 | 6.2 | 6.4 |
| SD | 0.2 | 0.0 | 0.3 | 0.2 | 0.1 | 0.1 | 0.4 |
| 1-Test-3 | 7.2 | 6.8 | 7.0 | 7.0 | 6.5 | 6.5 | 6.4 |
| 2-Test-1 | 6.9 | 6.5 | 6.4 | 6.4 | 6.3 | 6.3 | 6.1 |
| 4-Test-2 | 6.8 | ND | 6.4 | 6.4 | 6.4 | 6.3 | 6.3 |
| 5-Test-1 | 6.9 | 6.6 | 6.5 | 6.4 | 6.5 | 6.4 | 6.3 |
| Average | 6.9 | 6.7 | 6.6 | 6.6 | 6.4 | 6.4 | 6.3 |
| SD | 0.2 | 0.1 | 0.3 | 0.3 | 0.1 | 0.1 | 0.1 |

TABLE 14
Total Hemoglobin of RBCC during the storage at 1-6° C.

| | Total Hemoglobin, g/dL | | | | | | |
|---|---|---|---|---|---|---|---|
| Experiment- | | | | 21 | 28 | 35 | 42 |
| RBC unit | Pre-treat. | 7 days | 14 days | days | days | days | days |
| 1-Control | 16.0 | 14.5 | 14.9 | 14.5 | 14.8 | 14.8 | 15.2 |
| 2-Control | 17.0 | 15.4 | 15.1 | 15.2 | 15.0 | 15.3 | 15.4 |
| 4-Control | 16.2 | ND | 15.1 | 14.7 | 14.6 | ND | 14.7 |
| 5-Control | 16.5 | 14.6 | 15.7 | 14.8 | 14.3 | 15.3 | 14.6 |
| Average | 16.4 | 14.8 | 15.2 | 14.8 | 14.7 | 15.1 | 15.0 |
| SD | 0.4 | 0.5 | 0.3 | 0.3 | 0.3 | 0.3 | 0.4 |
| 1-Test-3 | 16.0 | 15.3 | 15.8 | 15.4 | 15.4 | 15.8 | 14.9 |
| 2-Test-1 | 17.0 | 15.7 | 15.0 | 14.1 | 15.3 | 15.2 | 15.6 |
| 4-Test-2 | 16.2 | ND | 15.4 | 15.0 | 15.2 | ND | 15.3 |
| 5-Test-1 | 16.5 | 15.7 | 16.0 | 16.3 | 16.0 | 15.9 | 15.7 |
| Average | 16.4 | 15.6 | 15.6 | 15.2 | 15.5 | 15.6 | 15.4 |
| SD | 0.4 | 0.2 | 0.4 | 0.9 | 0.4 | 0.4 | 0.4 |

TABLE 15
Mean Cellular Hemoglobin Concentration (MCHC) in
RBC during the storage at 1-6° C.

| | MCHC, g/dL | | | | | | |
|---|---|---|---|---|---|---|---|
| Experiment- | | | | 21 | 28 | 35 | 42 |
| RBC unit | Pre-treat. | 7 days | 14 days | days | days | days | days |
| 1-Control | 32.0 | 32.4 | 32.4 | 31.0 | 31.6 | 31.3 | 33.4 |
| 2-Control | 32.1 | 32.3 | 31.5 | 31.0 | 30.3 | 30.7 | 31.7 |
| 4-Control | 31.7 | ND | 30.8 | 31.5 | 30.5 | ND | 30.5 |
| 5-Control | 32.1 | 31.4 | 32.3 | 31.6 | 32.8 | 32.4 | 31.8 |
| Average | 32.0 | 32.0 | 31.8 | 31.3 | 31.3 | 31.5 | 31.9 |
| SD | 0.2 | 0.6 | 0.8 | 0.3 | 1.2 | 0.9 | 1.2 |
| 1-Test-3 | 32.0 | 32.7 | 33.2 | 33.3 | 32.7 | 32.6 | 32.4 |
| 2-Test-1 | 32.1 | 33.2 | 32.1 | 31.9 | 31.6 | 31.5 | 32.0 |
| 4-Test-2 | 31.7 | ND | 31.4 | 31.8 | 32.4 | ND | 31.8 |
| 5-Test-1 | 32.1 | 31.9 | 32.6 | 32.8 | 33.3 | 32.4 | 32.4 |
| Average | 32.0 | 32.6 | 32.3 | 32.5 | 32.5 | 32.2 | 32.2 |
| SD | 0.2 | 0.7 | 0.8 | 0.7 | 0.7 | 0.6 | 0.3 |

TABLE 16
Osmolality of RBCC during the storage at 1-6° C.

| | Osmolality, mOsm | | | | | | |
|---|---|---|---|---|---|---|---|
| Experiment- | | | | 21 | 28 | 35 | 42 |
| RBC unit | Pre-treat. | 7 days | 14 days | days | days | days | days |
| 1-Control | 350 | 288 | 285 | 287 | 285 | 293 | 287 |
| 2-Control | 365 | 284 | 288 | 289 | 292 | 291 | 296 |
| 4-Control | 341 | ND | 286 | 287 | 289 | 290 | 290 |
| 5-Control | 360 | 289 | 287 | 288 | 290 | 288 | 290 |
| Average | 354 | 287 | 287 | 288 | 289 | 291 | 291 |
| SD | 11 | 3 | 1 | 1 | 3 | 2 | 4 |
| 1-Test-3 | 350 | 290 | 288 | 287 | 290 | 298 | 292 |
| 2-Test-1 | 365 | 292 | 293 | 293 | 300 | 299 | 302 |
| 4-Test-2 | 341 | ND | 286 | 287 | 289 | 290 | 290 |
| 5-Test-1 | 360 | 292 | 290 | 291 | 293 | 291 | 299 |
| Average | 354 | 291 | 289 | 290 | 293 | 295 | 296 |
| SD | 11 | 1 | 3 | 3 | 5 | 5 | 6 |

TABLE 17
RBC count in RBCC duringthe storage at 1-6° C.

| | RBC count, 10⁶/□l | | | | | | |
|---|---|---|---|---|---|---|---|
| Experiment- | | | | 21 | 28 | 35 | 42 |
| RBC unit | Pre-treat. | 7 days | 14 days | days | days | days | days |
| 1-Control | 5.96 | 5.41 | 5.49 | 5.47 | 5.57 | 5.63 | 5.56 |
| 2-Control | 6.19 | 5.43 | 5.39 | 5.47 | 5.53 | 5.53 | 5.55 |
| 4-Control | 6.70 | ND | 6.18 | 6.09 | 6.09 | ND | 6.02 |
| 5-Control | 5.94 | 5.15 | 5.60 | 5.25 | 5.07 | 5.31 | 5.15 |
| Average | 6.20 | 5.33 | 5.67 | 5.57 | 5.57 | 5.49 | 5.57 |
| SD | 0.35 | 0.16 | 0.35 | 0.36 | 0.42 | 0.16 | 0.36 |
| 1-Test-3 | 5.96 | 5.68 | 5.86 | 5.64 | 5.69 | 5.83 | 5.64 |
| 2-Test-1 | 6.19 | 5.56 | 5.46 | 5.41 | 5.53 | 5.47 | 5.62 |
| 4-Test-2 | 6.70 | ND | 6.44 | 6.18 | 6.09 | ND | 6.15 |
| 5-Test-1 | 5.94 | 5.63 | 5.62 | 5.68 | 5.62 | 5.61 | 5.52 |
| Average | 6.20 | 5.62 | 5.85 | 5.73 | 5.73 | 5.64 | 5.73 |
| SD | 0.35 | 0.06 | 0.43 | 0.32 | 0.25 | 0.18 | 0.28 |

The average hemolysis level was higher in RBCC units purified by automated washing (Control set) than in units purified using CE columns (Test set) during the whole period of storage (0.46±0.14% vs. 0.29±0.08% after 42 days of storage). However, even after 42 days of storage, the average level of hemolysis was below 1% for both sets.

The average ATP level was slightly better preserved in Test units than in Control units (2.78±0.42 vs. 1.61±0.08 μmole/g Hgb respectively) after 42 days of storage.

The other parameters measured in the study were almost identical for Control and Test sets.

Conclusions

1. The efficiency of PEN110 removal from RBCC using DOWEX 50W×8 columns depended on 1) the size of the column (the amount of resin in the column); 2) the flow rate; 3) the length of the column and 4) the temperature. Of the foregoing parameters, the efficiency of PEN110 removal using DOWEX 50W×8 columns increased with an increase in 1) the size of the column (the amount of resin in the column); 2) the length of the column and 3) the temperature. The efficiency of PEN110 removal decreased with an increase in flow rate.

2. Manual washing typically provided an additional tenfold decrease in post-column PEN110 concentration.

3. Post-column reduction of PEN110 concentration to a level below 50 ng/mL was achieved when RBCC units were passed through 2.5×75 cm DOWEX 50W×8 columns at 25-27° C. with average flow rate 7-8 mL/min.

4. Post-manual wash reduction of PEN110 concentration to a level below 50 ng/mL was achieved when RBCC units were passed through 2.5×30 cm DOWEX 50W×8 columns at 25-27° C. with average flow rate 1-2 mL/min.

5. The in vitro quality of RBCC purified using DOWEX 50W×8 columns was almost identical to the quality of RBCC purified by automated washing.

### Example 2

### Comparison of Strong Cation Exchanger vs. Weak Cation Exchangers for Removal of Ethyleneimine Oligomer from Red Blood Cell Concentrates

The purpose of the experiment was to compare the efficiency of PEN110 removal from RBCC with strong cation exchanger (DOWEX 50W×8) versus with weak cation exchangers (DOWEX MAC3 and PUROLITE C115).

Methods:

PEN110 removal from RBCC was assessed using Dowex MAC3, Purolite C115 and Dowex 50W×8 2.5×20 cm cartridges. Three identical units of PEN110 treated RBCC were purified using 2.5×20 cm DOWEX 50W×8 (strong cation exchanger) and Dowex MAC 3 and Purolite C115 (weak cation exchangers) with flow rate 3-4 ml/min at 26±1° C. using a methodology similar to that described in Example 1, above. The column flow rate was controlled by clamping the line between the incubation bag and the column.

Preparation of Resins in Na⁺ Form:

830 g (approximately 1 L) of dry DOWEX 50W×8 resin in hydrogen form was suspended in 2 L of deionized water and gently mixed. Supernatant fluid was removed by decanting. The resin was washed again with 2 L of water and loaded on a 7.5×56 cm (2 L) glass column, with a sintered filter. The resin on the column was washed with ˜1.5 L of 1 M NaOH. The pH of the eluate was monitored using pH indicator strips. Washing was continued until the pH of eluate reached 13-14. The resin was then washed with deionized water (˜4 L) until the pH of the eluate decreased to 7. Finally, the resin was washed with 2 L of methanol, transferred to a 1 L thick-walled plastic container, and dried under reduced pressure (6 mm Hg) for 24 hrs at room temperature.

Approximately 200 g (1 L wet resin) of DOWEX MAC2 and PUROLITE C115 resins were transferred into Na⁺ using the approach described above for the DOWEX 50W×8 resin. The parameters of the weak cation exchange resins used in this experiment are shown in Table 18, below:

TABLE 18
Bead Parameters

| Name of beads | DOWEX MAC3 | PUROLITE C115 |
|---|---|---|
| Matrix composition | Polyacrylic, | Polymethacrylic, |
| | macroporous | macroporous |
| Size of beads (mesh) | 16-50 mesh | 16-50 mesh |
| Cation exchange group | Carboxyl | Carboxyl |
| Ionic form | Na⁺ | Na⁺ |
| Total exchange capacity | 3.8 | 3.5 |
| mEq/ml | | |
| Operating pH range | 4-14 | 5-15 |
| Volume of 1 g dry resin (ml) | 2.2 | 2.1 |
| Swelling in saline | 3 times | 3 times |

Preparation of Cartridges

The suspensions of resins in saline (1 g DOWEX 50W×8 dry resin in 2.5 ml and 1 g DOWEX MAC3 or PUROLITE C115 dry resin in 10 ml saline) were prepared. Suspended beacs were loaded into 2.5×20 Flex-Columns (Kontes, Inc.). Two C115 and one MAC3 and 50W×8 columns were prepared. Final volumes and weights of used resin are shown in Table 18. All cartridges were flushes with ˜500 ml of saline prior to use.

PEN110 Treatment of RBCC

Four compatible units of CPD/AS-1 RBCC were pooled together as described above and injected with PEN110 to a final concentration of 0.1% (v/v). The PEN110 was delivered to the pool as a neutral 2% (v/v) stock solution in 0.25 M filter-sterilized NaH₂PO₄. Immediately after PEN110 delivery, the RBCC pool was split into four identical units in 600 mL transfer packs (Baxter). The units were incubated for 24 hr at 23° C. Samples were collected at 0 and 24 hours (T0 and T24, respectively) for HPLC analysis.

Solid Phase Extraction

RBCC units were passed through the columns with 3-4 ml/min flow rate at 25-27° C. and collected in 600 mL transfer packs. Post column HPLC samples were collected.

PEN110 Quantitation

T0 and T24 samples for PEN110 quantitation were prepared by mixing 50 μL of RBCC with 1450 μL of water (dilution 1:30); for the preparation of the post column samples, 200 μL of RBCC were mixed with 800 μL of water (1:5 dilution). The residual level of PEN110 in RBCC was determined by cation-exchange HPLC coupled with post-column OPA derivatization following QCP-W003: HPLC analysis of PEN110 in RBC lysates, low range, as described above.

Results:

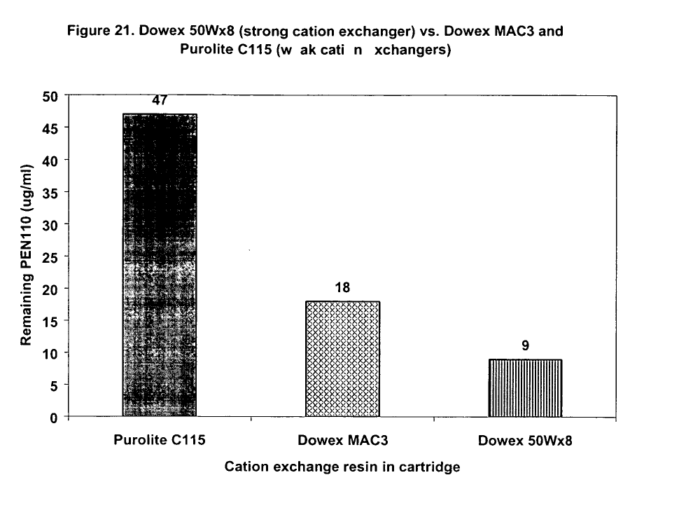

The experimental conditions and PEN110 quantitation results by HPLC are shown in Table 19 and FIG. 21.

TABLE 19
PEN110 removal from RBCC using 2.5 × 20 cm Dowex 50x8,
Dowex MAC3 and Purolite C115 columns: Parameters of columns tested,
experimental conditions and PEN110 quantitation results by HPLC.

| | Experimental parameters | | | | | | | PEN110 concentration | | |
|---|---|---|---|---|---|---|---|---|---|---|
| | | Volume | | | | | | | | Post |
| | Column | of wet | Weight of | Average | | RBCC | | | | Column |
| Type of | size, | resin, | dry resin, | flow rate, | RT, | volume, | Hct, | T0 HPLC | T24 HPLC | HPLC |
| cartridge | cm | mL | g | mL/min | ° C. | mL | % | μg/mL | μg/mL | μg/mL |
| Purolite | 2.5 × 20 | 100 | 20 | 3-4 | 26.6 | 346 | 58 | 935 | 518 | 46 |
| C115 #1 | | | | | | | | | | |
| Dowex | | | 20 | | | 346 | | | | 18 |
| MAC3 | | | | | | | | | | |
| Dowex | | | 50 | | | 349 | | | | 9 |
| 50Wx8 | | | | | | | | | | |
| Purolite | | | 20 | | | 350 | | | | 49 |
| C115 #2 | | | | | | | | | | |

Note:
Purolite C115 cartridges #1 and #2 were duplicates.

Conclusions

Weak cation exchangers can be employed for the removal of PEN110 from RBCC units.

The removal efficiency of PEN110 from RBCC with weak cation exchange columns (Purolite C115 and Dowex MAC3) was not as effective as with a strong cation exchange column (Dowex 50W×8).

### Example 3

### Stepwise Removal of PEN110 from RBCC with Fresh Portions of Dowex 50W×8 Beads

The purpose of the experiment was to determine the feasibility of stepwise removal of PEN110 from RBCC to a level below 50 ng/ml by direct presentation with fresh portions of Dowex 50W×8 beads followed with two steps of manual washing.

Methods:

Stepwise Removal of PEN110 from RBCC with Fresh Portions of Dowex Beads:

27 g of DOWEX 50W×8 beads were loaded into 600 ml RBCC transferring bags (Baxter) and suspended in 60 ml of saline. Five transferring bags with Dowex beads were used. A unit of PEN110 treated RBCC was transferred stepwise from bag to bag containing Dowex beads, and each time gently agitated for 45 min. After the last transferring step, the treated RBCC was manually washed as described above.

Results:

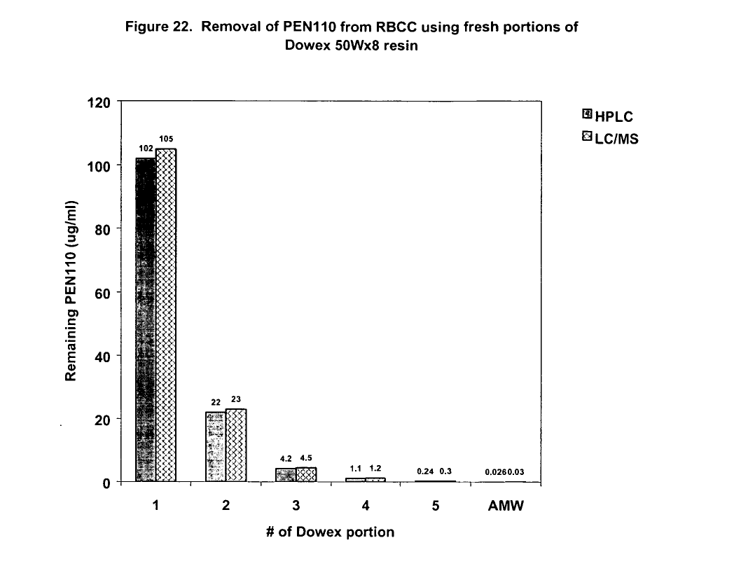

Experimental conditions and PEN110 quantitation results by HPLC and LC/MS are shown in Table 20 and FIG. 22.

TABLE 20
PEN110 removal from RBCC using fresh portions of
Dowex 50x8 loaded in transfer bags: Experimental
conditions and PEN110 quantitation results.

| | Experimental | | | | |
|---|---|---|---|---|---|
| | parameters | | | | |
| | Weight of | Incubation | PEN110 concentration | | |
| RBCC | dry resin, | time, | T24 HPLC | HPLC, | LC/MS, |
| treatment | g | min | μg/mL | μg/mL | μg/mL |
| Portion #1 | 27 | 45 | 799 | 102 | 105 |
| Portion #2 | | | | 22 | 23 |
| Portion #3 | | | | 4.2 | 4.5 |
| Portion #4 | | | | 1.1 | 1.2 |
| Portion #5 | | | | 0.24 | 0.3 |
| After manual | | | | 0.026 | 0.03 |
| washing | | | | | |

Conclusion

Removal of PEN110 from RBCC unit to a level below 50 ng/ml was achieved by stepwise incubation of RBCC with fresh Dowex beads followed by two cycles of manual washing.

References

1. Weber W. J., Jr., Physicochemical Processes for Water Quality Control, Wiley Interscience, 1972.

2. Dechow F. J., Separation and Purification Techniques in Biotechnology, Noyes Publication, 1989.

3. Inaba S., Keiko K., Takano H., Maeda Y., Uehara K., Oshige T., Yuasa T. and Nakashima H., Transfusion, 2000;40:1469-1474.

4. Burstain J. M., Brecher M. E., Halling V., Pineda A. A. Blood volume determination as a function of hematocrit and mass in three preservative solutions and saline., Am. J. Clin. Pathol., 1994;102, 812-815.

Equivalents

Those skilled in the art will recognize, or be able to ascertain using no more than routine experimentation, many equivalents to the specific embodiments of the invention described herein. Such equivalents are intended to be encompassed by the following claims.

All patents, patent applications and references disclosed herein are incorporated by reference in their entirety.

We claim:

1. A method removing a positively charged microbicidal compound and/or degradation products or derivatives thereof from a composition, comprising
contacting a composition with a cation exchange resin, under conditions and for a time sufficient to allow a positively charged microbicidal compound and/or degradation products or derivatives thereof in the composition to bind to the cation exchange resin, and
separating the positively charged microbicidal compound and/or degradation products or derivatives thereof bound to the cation exchange resin from the composition.

2. The method of claim 1, wherein the positively charged microbicidal compound is positively charged at physiological pH.

3. The method of claim 1, wherein the cation exchange resin is not contained within a matrix.

4. The method of claim 1, wherein the positively charged microbicidal compound is an aziridino compound.

5. The method of claim 4, wherein the aziridino compound is an ethyleneimine oligomer.

6. The method of claim 1, wherein the composition is a blood product.

7. The method of claims 6, wherein the method does not substantially change the biological properties of the treated blood product relative to untreated blood product.

8. The method of claim 7, wherein the biological properties of the treated blood product are not substantially changed after storage at 4° C. relative to untreated blood product.

9. The method of claim 6, wherein the blood product is a composition comprising red blood cells.

10. The method of claim 9, wherein the method results in a level of hemolysis of the red blood cells that is no greater than cell washing

11. The method of claim 9, wherein the surface of the cation exchange resin particles does not induce substantial hemolysis of the red blood cells.

12. The method of claim 6, further comprising washing the blood product after removal of the positively charged microbicidal compound bound to the cation exchange resin.

13. The method of claim 6, further comprising washing the blood product prior to contacting the composition with a cation exchange resin.

14. The method of claim 1, wherein the cation exchange resin is a strongly acidic cation exchange resin.

15. The method of claim 1, wherein the cation exchange resin comprises sulfonic groups.

16. The method of claim 1, wherein the diameter of the cation exchange resin particles is at least about 100 microns.

17. The method of claim 16, wherein the size of the particles is between about 150 microns and about 300 microns.

18. The method of claim 1, wherein the cation exchange resin particles are substantially non-breakable under moderate mechanical stress in dry conditions or suspensions.

19. The method of claim 1, wherein the cation exchange resin particles are compatible with water miscible solvents.

20. The method of claim 19, wherein the cation exchange resin particles do not dissolve or degrade when contacted with water miscible solvents.

21. The method of claim 1, wherein the cation exchange resin particles do not create fine particles under moderate mechanical stress.

22. The method of claim 1, wherein the cation exchange resin particles have a cation exchange capacity of at least about 1 meq/ml.

23. The method of claim 22, wherein the cation exchange resin particles have a cation exchange capacity of at least about 2 meq/ml.

24. The method of claim 22, wherein the cation exchange resin particles have a cation exchange capacity of at least about 3 meq/ml.

25. The method of claim 24, wherein the cation exchange resin particles have a cation exchange capacity of at least about 5 meq/ml.

26. The method of claim 1, wherein the method is performed under pH conditions of from about pH 4 to about pH 14.

27. The method of claim 26, wherein the method is performed under pH conditions of from about pH 6 to about pH 8.

28. The method of claim 27, wherein the method is performed at about pH 7.

29. The method of claim 1, wherein the cation exchange resin particles do not leach toxic components into water based media or blood products.

30. The method of claim 1, wherein the cation exchange resin particles are sterilized by gamma or thermal sterilization.

31. The method of claim 1, wherein the cation exchange resin is DOWEX™ 50W×8.

32. The method of claim 1, wherein the method is performed using a column format, in which the step of contacting a composition with a cation exchange resin is performed by flowing the composition into a column packed with the cation exchange resin, and the step of separating the positively charged microbicidal compound and/or degradation products or derivatives thereof bound to the cation exchange resin from the composition is performed by flowing the composition out of the column.

33. The method of claim 32, wherein the column is run by gravity or by moderate pressure with a flow rate of less than about 50 ml/min.

34. The method of claim 33, wherein the flow rate is less than about 10 ml/min.

35. The method of claim 32, wherein the flow rate is less than about 1 ml/min.

36. The method of claim 1, wherein the cation exchange resin particles have substantially no pores.

37. The method of claim 1, wherein the amount of positively charged microbicidal compound in the composition is reduced by at least about 2 logs.

38. The method of claim 37, wherein the amount of positively charged microbicidal compound in the composition is reduced by at least about 3 logs.

39. The method of claim 38, wherein the amount of positively charged microbicidal compound in the composition is reduced by at least about 4 logs.

40. The method of claim 39, wherein the amount of positively charged microbicidal compound in the composition is reduced by at least about 5 logs.

41. The method of claim 1, wherein the method is performed at a temperature of at least about 20° C.

42. The method of claim 1, wherein the method is performed at a temperature of at least about 25° C.

43. The method of claim 1, wherein the method is performed at a temperature of at least about 27° C.

44. The method of claim 1, wherein the method is performed at a temperature of at least about 30° C.

45. The method of claim 32, wherein the dimensions of the column expressed as a ratio of diameter:length are about 1:5 or less.

46. The method of claim 45, wherein the dimensions of the column expressed as a ratio of diameter:length are about 1:10 or less.

47. The method of claim 45, wherein the dimensions of the column expressed as a ratio of diameter:length are about 1:20 or less.

48. The method of claim 1, wherein method is performed in a column format, the flow rate is from about 1 ml/min to about 2 ml/min, the temperature is about 25° C., and the dimensions of the column expressed as a ratio of diameter:length are about 1:10 or less.

49. The method of claim 1, wherein method is performed in a batch format.

50. The method of claim 49, wherein the step of separating the positively charged microbicidal compound and/or degradation products or derivatives thereof bound to the cation exchange resin from the composition is performed by filtration of the composition to remove the cation exhange resin.

51. The method of claim 49, wherein the step of separating the positively charged microbicidal compound and/or degradation products or derivatives thereof bound to the cation exchange resin from the composition is performed by centrifugation of the composition to remove the cation exchange resin.

52. The method of claim 49, wherein the step of contacting the composition with the cation exchange resin is performed by adding the cation exchange resin in a permeable container to the composition.

53. The method of claim 52, wherein the step of separating the positively charged microbicidal compound and/or degradation products or derivatives thereof bound to the cation exchange resin from the composition is performed by removing the permeable container from the composition.

54. The method of claim 49, wherein the step of contacting the composition with the cation exchange resin is performed by adding the composition to a container that contains cation exchange resin.

55. The method of claim 54, wherein the step of separating the positively charged microbicidal compound and/or degradation products or derivatives thereof bound to the cation exchange resin from the composition is performed by removing the composition from the container.

56. The method of claim 54, wherein the step of separating the positively charged microbicidal compound and/or degradation products or derivatives thereof bound to the cation exchange resin from the composition is performed by removing the resin and bound positively charged microbicidal compound and/or degradation products or derivatives thereof from the container.

57. The method of claim 49, wherein the step of contacting the composition with the cation exchange resin is performed by adding the cation exchange resin to the composition.

58. The method of claim 57, wherein the step of separating the positively charged microbicidal compound and/or degradation products or derivatives thereof bound to the cation exchange resin from the composition is performed by filtration of the composition to remove the cation exhange resin.

59. The method of claim 57, wherein the step of separating the positively charged microbicidal compound and/or degradation products or derivatives thereof bound to the cation exchange resin from the composition is performed by centrifugation of the composition to remove the cation exchange resin.

60. The method of claim 49-59, wherein the steps of contacting the composition with the cation exchange resin and separating the positively charged microbicidal compound and/or degradation products or derivatives thereof bound to the cation exchange resin from the composition are performed at least twice.

61. The method of any of claims 49-60, wherein the composition comprises cells, further comprising washing the cells.

62. The method of claim 61, wherein the cells are washed using an automated cell washer.

63. The method of any of claims 1-62, wherein the concentration of microbicidal compound is reduced by at least about 50%.

64. The method of claim 63, wherein the concentration of microbicidal compound is reduced by at least about 1 log.

65. The method of claim 64, wherein the concentration of microbicidal compound is reduced by at least about 2 logs.

66. The method of claim 65, wherein the concentration of microbicidal compound is reduced by at least about 3 logs.

67. The method of claim 66, wherein the concentration of microbicidal compound is reduced by at least about 4 logs.

68. A method for removal of an intracellular positively charged microbicidal compound and/or degradation products or derivatives thereof from a cell treated with a positively charged microbicidal compound, comprising
contacting a composition containing a cell treated with a positively charged microbicidal compound with a cation exchange resin, under conditions and for a time sufficient to allow the positively charged microbicidal compound and/or degradation products or derivatives thereof to bind to the cation exchange resin.

69. The method of claim 68, further comprising a step of separating the positively charged microbicidal compound and/or degradation products or derivatives thereof bound to the cation exchange resin from the cell.

70. The method of claim 68, wherein the positively charged microbicidal compound is positively charged at physiological pH.

71. The method of claim 68, wherein the cation exchange resin is not contained within a matrix.

72. The method of claim 68, wherein the positively charged microbicidal compound is an aziridino compound.

73. The method of claim 72, wherein the aziridino compound is an ethyleneimine oligomer.

74. The method of claim 68, wherein the composition is a blood product.

75. The method of claims 74, wherein the method does not substantially change the biological properties of the treated blood product relative to untreated blood product.

76. The method of claim 75, wherein the biological properties of the treated blood product are not substantially changed after storage at 4° C. relative to untreated blood product.

77. The method of claim 74, wherein the blood product is a composition comprising red blood cells.

78. The method of claim 77, wherein the method results in a level of hemolysis of the red blood cells that is no greater than cell washing

79. The method of claim 77, wherein the surface of the cation exchange resin particles does not induce substantial hemolysis of the red blood cells.

80. The method of claim 74, further comprising washing the blood product after removal of the positively charged microbicidal compound bound to the cation exchange resin.

81. The method of claim 74, further comprising washing the blood product prior to contacting the composition with a cation exchange resin.

82. The method of claim 68, wherein the cation exchange resin is a strongly acidic cation exchange resin.

83. The method of claim 68, wherein the cation exchange resin comprises sulfonic groups.

84. The method of claim 68, wherein the diameter of the cation exchange resin particles is at least about 100 microns.

85. The method of claim 84, wherein the size of the particles is between about 150 microns and about 300 microns.

86. The method of claim 68, wherein the cation exchange resin particles are substantially non-breakable under moderate mechanical stress in dry conditions or suspensions.

87. The method of claim 68, wherein the cation exchange resin particles are compatible with water miscible solvents.

88. The method of claim 87, wherein the cation exchange resin particles do not dissolve or degrade when contacted with water miscible solvents.

89. The method of claim 68, wherein the cation exchange resin particles do not create fine particles under moderate mechanical stress.

90. The method of claim 68, wherein the cation exchange resin particles have a cation exchange capacity of at least about 1 meq/ml.

91. The method of claim 90, wherein the cation exchange resin particles have a cation exchange capacity of at least about 2 meq/ml.

92. The method of claim 90, wherein the cation exchange resin particles have a cation exchange capacity of at least about 3 meq/ml.

93. The method of claim 92, wherein the cation exchange resin particles have a cation exchange capacity of at least about 5 meq/ml.

94. The method of claim 68, wherein the method is performed under pH conditions of from about pH 4 to about pH 14.

95. The method of claim 94, wherein the method is performed under pH conditions of from about pH 6 to about pH 8.

96. The method of claim 95, wherein the method is performed at about pH 7.

97. The method of claim 68, wherein the cation exchange resin particles do not leach toxic components into water based media or blood products.

98. The method of claim 68, wherein the cation exchange resin particles are sterilized by gamma or thermal sterilization.

99. The method of claim 68, wherein the cation exchange resin is DOWEX™ 50W×8.

100. The method of claim 68, wherein the method is performed using a column format, in which the step of contacting a composition with a cation exchange resin is performed by flowing the composition into a column packed with the cation exchange resin, and the step of separating the positively charged microbicidal compound and/or degradation products or derivatives thereof bound to the cation exchange resin from the composition is performed by flowing the composition out of the column.

101. The method of claim 100, wherein the column is run by gravity or by moderate pressure with a flow rate of less than about 50 ml/min.

102. The method of claim 101, wherein the flow rate is less than about 10 ml/min.

103. The method of claim 100, wherein the flow rate is less than about 1 ml/min.

104. The method of claim 68, wherein the cation exchange resin particles have substantially no pores.

105. The method of claim 68, wherein the amount of positively charged microbicidal compound in the composition is reduced by at least about 2 logs.

106. The method of claim 105, wherein the amount of positively charged microbicidal compound in the composition is reduced by at least about 3 logs.

107. The method of claim 106, wherein the amount of positively charged microbicidal compound in the composition is reduced by at least about 4 logs.

108. The method of claim 107, wherein the amount of positively charged microbicidal compound in the composition is reduced by at least about 5 logs.

109. The method of claim 68, wherein the method is performed at a temperature of at least about 20° C.

110. The method of claim 68, wherein the method is performed at a temperature of at least about 25° C.

111. The method of claim 68, wherein the method is performed at a temperature of at least about 27° C.

112. The method of claim 68, wherein the method is performed at a temperature of at least about 30° C.

113. The method of claim 100, wherein the dimensions of the column expressed as a ratio of diameter:length are about 1:5 or less.

114. The method of claim 113, wherein the dimensions of the column expressed as a ratio of diameter:length are about 1:10 or less.

115. The method of claim 113, wherein the dimensions of the column expressed as a ratio of diameter:length are about 1:20 or less.

116. The method of claim 68, wherein method is performed in a column format, the flow rate is from about 1 ml/min to about 2 ml/min, the temperature is about 25° C., and the dimensions of the column expressed as a ratio of diameter:length are about 1:10 or less.

117. A blood product treated according to the method of any of claims 1-116.

118. A container for blood product comprising cation exchange resin.

119. The container of claim 118, wherein the cation exchange resin is loose.

120. The container of claim 118, wherein the cation exchange resin is contained within a permeable enclosure.

121. The container of claim 118, wherein the cation exchange resin is a strong cation exchanger.

122. The container of claim 118, wherein the amount of cation exchange resin is sufficient to reduce the amount of a positively charged microbicidal compound and/or degradation products or derivatives thereof in the blood product after contact with the resin.

123. The container of claim 122, wherein the amount of cation exchange resin is sufficient to reduce the amount of the positively charged microbicidal compound and/or degradation products or derivatives thereof by at least about 50%.

124. The container of claim 122 or 123, wherein the positively charged microbicidal compound is an ethyleneimine oligomer.
